(19) United States
(12) Patent Application Publication
(10) Pub. No.: US 2005/0182250 A1
(43) Pub. Date: Aug. 18, 2005

(54) Humanized renilla reniformis green fluorescent protein as a scaffold

(75) Inventors: Scott Happe, Austin, TX (US); Dwight Dubois, Austin, TX (US); Katie J. Leininger, New York, NY (US)

Correspondence Address: PALMER & DODGE, LLP;KATHLEEN M. WILLIAMS / STR, 111 HUNTINGTON AVENUE, BOSTON, MA, 02199, US

(73) Assignee: Stratagene

(21) Appl. No.: 10/615,064

(22) Filed: Jul. 8, 2003

Related U.S. Application Data
(60) Provisional application No. 60/394,737, filed on Jul. 10, 2002.

(51) Int. Cl. C12Q 1/68; C07H 21/04; C07K 14/435

(52) U.S. Cl. 536/23.5; 435/69.1; 435/320.1; 435/325; 530/350; 436/86; 435/6

(57) ABSTRACT

The present invention discloses green fluorescent protein (GFP) and GFP variants that are derived from Renilla reniformis. The Renilla reniformis GFP and variants there of, are optimized for expression in human cells and are further used as a scaffold for the in vivo display of peptides and peptide libraries.

### RELATED APPLICATIONS

This application claims the priority of U.S. Provisional Application No. 60/394,737, filed Jul. 10, 2002, the entirety of which is incorporated herein by reference, including figures.

### FIELD OF THE INVENTION

The present invention relates to humanized Renilla reniformis green fluorescent protein (hrGFP) and its use as a protein scaffold for the presentation of functional peptides.

### BACKGROUND OF THE INVENTION

Green fluorescent protein (GFP) from Aequorea Victoria has been used as a scaffold for the in vivo display of peptides and peptide libraries in both yeast and mammalian cells (Kamb et al. (1998) Proc. Natl. Acad. Sci. USA, 95: 7508-7513). GFP as a protein scaffold for the display of random peptides may be used to define the characteristics of a peptide library. For example, Abedi et al (1998, Nucleic Acids Res. 26: 623-300) have inserted peptides into the solvent-exposed looped regions of Aequorea victoria GFP and show that the GFP molecules retain their autofluorescence when expressed in yeast and Escherichia coli. Abedi et al. further show that the fluorescence of the GFP scaffold can be used to monitor peptide diversity, as well as the presence, or expression of a peptide in a given cell. However, the mean fluoresence of the GFP scaffold molecules is relatively low in comparison with wt GFP. Kamb and Abedi (U.S. Pat. No. 6,025,485) have prepared GFP scaffold libraries with enhanced green fluorescent protein (EGFP) in order to enhance the fluoresence intensity. In addition, Peelle et al. (2001, Chem. & Bio. 8: 521-534) has recently tested EGFP scaffold peptide libraries with different structural biases in mammalian cells. Anderson et al. further improved on fluoresence intensity by insertion of peptides into GFP loops with tetraglycine linkers (U.S. Patent Aplication 2001/0003650). However, there is a need in the art for GFP scaffolds that not only exhibit optimal fluoresence, but also GFP scaffolds that can be expressed at high levels within cells. There exists variability among GFPs in the tolerance for display while retaining autofluorescence, and thus there also is a need in the art for GFPs that can be expressed at high levels and tolerate insertions while preserving GFP autofluorescence.

### SUMMARY OF THE INVENTION

The present invention discloses green fluorescent protein (GFP) and GFP variants derived from Renilla reniformis that are both optimized for expression in human cells and that are useful as a scaffold for the in vivo display of peptides and peptide libraries.

The invention encompasses a recombinant polynucleotide comprising a first nucleic acid sequence encoding humanized Renilla reniformis green fluorescent protein (hrGFP) and a second heterologous nucleic acid sequence inserted internally into said first nucleic acid sequence encoding humanized hrGFP.

In one embodiment, the recombinant polynucleotide comprises the sequence identified in SEQ ID NO: 1.

In another embodiment the recombinant polynucleotide comprises a heterologous nucleic acid sequence is inserted between nucleotides 519 and 520 of the nucleic acid sequence encoding hrGFP.

The invention further encompasses a recombinant polynucleotide wherein the heterologous nucleic acid sequence is a multiple cloning site sequence.

In one embodiment, the recombinant polynucleotide comprising the multiple cloning site is the sequence identified in SEQ ID NO: 2.

In an additional embodiment, the recombinant polynucleotide further comprises a third nucleic acid sequence inserted internally into a multiple cloning site, wherein the third nucleic acid sequence is a random nucleic acid sequence.

In one embodiment, the third nucleic acid sequence encodes a peptide in frame with hrGFP.

In another embodiment, the third nucleic acid sequence encodes a peptide of 2 to 50 amino acids. In a preferred embodiment, the third nucleic acid sequence encodes a peptide of about 10 to about 20 amino acids.

The invention also encompasses a recombinant polypeptide comprising Renilla reniformis green fluorescent protein (GFP) and a heterologous peptide that is fused internally into said GFP.

In one embodiment, the recombinant polypeptide comprises a heterologous peptide that is located between amino acid residues 173 and 174 of Renilla reniformis GFP.

In another embodiment, the recombinant polypeptide comprises a heterologous random peptide sequence.

The invention additionally encompasses recombinant vectors that comprise the above mentioned recombinant polynucleotides.

In one embodiment, the recombinant vector is selected from the group consisting of a plasmid, a bacteriophage, a virus, and a retrovirus.

The invention further encompasses cells that comprise the recombinant vectors comprising the recombinant polynucleotides that comprise a first nucleic acid sequence encoding humanized Renilla reniformis green fluorescent protein (hrGFP) and a second heterologous nucleic acid sequence inserted internally into said first nucleic acid sequence encoding humanized hrGFP.

The invention further encompasses a library of recombinant vectors that contain recombinant polynucleotides, wherein the recombinant polynucleotides comprise a first nucleic acid sequence encoding Renilla reniformis green fluorescent protein (hrGFP) and a second heterologous random nucleic acid sequence inserted internally into the first nucleic acid sequence encoding hrGFP. The library comprises a plurality of recombinant vectors that differ in sequence by virtue of the random nucleic acid.

The invention provides for a method of identifying peptides that confer a phenotype of interest. The method comprises the steps of i) providing a plurality of cells that contain a recombinant vector that encodes a recombinant polypeptide of Renilla reniformis green fluorescent protein (hrGFP) and a heterologous random peptide that is fused internally into hrGFP, and i) assaying the cells for said phenotype.

The invention further provides a method to identify peptides that interact with a protein of interest. The method comprises introducing into host cells a library of recombinant vectors that encode recombinant polypeptides of Renilla reniformis green fluorescent protein (hrGFP) fused to a transactivation domain and a random heterologous peptide that is fused internally into hrGFP. In this method, the host cells contain a gene that encodes a protein of interest fused to a DNA binding domain and a reporter gene functionally linked to a DNA sequence bound by the DNA binding domain fusion protein. The expression of the reporter gene is regulated by the transactivation domain fusion protein and thus detection of reporter gene expression indicates that the peptide interacts with the protein of interest.

### BRIEF DESCRIPTION OF THE FIGURES

The objects and features of the invention can be better understood with reference to the following detailed description and accompanying drawings.

FIG. 1 is a nucleic acid sequence of humanized Renilla reniformis GFP (hrGFP)

FIG. 2 is the nucleic acid sequence of humanized Renilla reniformis GFP that has 18 nucleotide bases inserted between nucleotides 519 and 520 of hrGFP, hrpGFP-173. The insert comprises BglII, EcoRI, and AatII restriction enzyme recognition sequences. The 18 nucleotide insert is underlined and encodes a six amino acid insert between amino acids 173 and 174 of wild-type hrGFP.

FIG. 3 is the amino acid sequence of Renilla reniformis GFP.

FIG. 4 is the amino acid sequence of hrGFP-173. The six amino acid insert between amino acids 173 and 174 of wild-type hrGFP is underlined.

FIG. 5 shows the nucleic acid sequence of wild-type Renilla reniformis GFP.

FIG. 6 shows that the hrGFP-173 insertion mutant fluoresces in 293 cells. FIG. 6a shows fluorescence 24 h after transfection, and FIG. 6b (upper left panel, hrGFP-173) shows fluorescence approximately 70 hours after transfection.

FIG. 7 shows that the GFP-173 insertion mutant (upper left panel) qualitatively produces more fluorescence in comparison to wild-type hrGFP (lower right panel) than hrGFP-174 (upper right panel) and hrGFP 175 (lower left panel).

### DETAILED DESCRIPTION

The present invention relates to GFP and variants derived from Renilla reniformis that are both optimized for expression in human cells, and useful as a scaffold for the in vivo display of peptides and peptide libraries.

The present invention further discloses methods of using the humanized Renilla reniformis GFP peptide libraries to identify peptides that may be used for drug discovery or intracellular knock-out reagents.

Definitions

The following definitions are provided for specific terms which are used in the following written description.

As used herein, the term “humanized R. reniformis green fluorescent protein” or “R. reniformis GFP” refers to a polypeptide of SEQ ID NO: 3, or to a fluorescent variant thereof. An R. reniformis GFP variant encompasses polypeptides of SEQ ID NO: 4 that bear one or more mutations, including insertion or deletion of one or more amino acids, either at the N or C termini of the polypeptide or internal to the coding sequence. Variants of R. reniformis GFP according to the invention retain the ability to emit light when excited by light within a given part of the spectrum, and can be be excited by light of, or emit light in a portion of the spectrum that differs detectably from that which excites or which is emitted by wild-type R. reniformis. In addition to variants exhibiting different excitation or emission spectra, R. reniformis GFP variants include variants exhibiting increased fluorescence intensity relative to wild-type R. reniformis GFP.

The term “variant thereof” when used in reference to a “humanized” R. reniformis polynucleotide coding sequence means that the sequence bears one or more nucleotide differences relative to the sequence of the wild-type R. reniformis coding sequence of SEQ ID NO: 5. A variant of an R. reniformis polynucleotide sequence encodes an R. reniformis GFP polypeptide or a variant thereof. A variant polynucleotide directs the expression of an amount of fluorescent polypeptide at least equal to, or greater than, the amount expressed from an equal mass amount or from an equal number of copies of a non-humanized R. reniformis GFP polynucleotide sequence. As used herein, a variant polynucleotide is a “humanized polynucleotide”.

The term “humanized polynucleotide” or “humanized sequence” refers to a polynucleotide coding sequence in which one or more, including 5 or more, 10 or more, 20 or more, 50 or more, 75 or more, 100 or more, 125 or more, 150 or more, 200 or more, or even all codons of the polynucleotide coding sequence for a non-human polypeptide (i.e., a polypeptide not naturally expressed in humans) have been altered to a codon sequence more preferred for expression in human cells. Because there are 64 possible combinations of the 4 DNA nucleotides in codon groups of 3, the genetic code is redundant for many of the 20 amino acids. Each of the different codons for a given amino acid encodes the incorporation of that amino acid into a polypeptide. However, within a given species there tends to be a preference for certain of the redundant codons to encode a given amino acid. The “codon preference” of R. reniformis is different from that of humans (this codon preference is usually based upon differences in the level of expression of the tRNAs containing the corresponding anticodon sequences). In order to obtain high expression of a non-human gene product in human cells, it is advantageous to change one or more non-preferred codons to a codon sequence that is preferred in human cells. Table 1 shows the preferred codons for human gene expression. A codon sequence is preferred for human expression if it occurs to the left of a given codon sequence in the table. Optimally, but not necessarily, less preferred codons in a non-human polynucleotide coding sequence are humanized by altering them to the codon most preferred for that amino acid in human gene expression. The amount of fluorescent polypeptide expressed in a human cell from a humanized GFP polynucleotide sequence according to the invention is at least two-fold greater, on either a mass or a fluorescence intensity scale per cell, than the amount expressed from an equal amount or number of copies of a non-humanized GFP polynucleotide.

As used herein, the term “humanized codon” means a codon sequence, within a polynucleotide sequence encoding a non-human polypeptide, that has been changed to a codon sequence that is more preferred for expression in human cells relative to that codon encoded by the non-human organism from which the non-human polypeptide is derived. “Preferred” codons have a greater pool of tRNA molecules to use during expression than non-preferred codons, for example the tRNA molecules are not limiting for expression of a particular polypeptide. Species-specific codon preferences stem in part from differences in the expression of tRNA molecules with the appropriate anticodon sequence. That is, one factor in the species-specific codon preference is the realtionship between a codon and the amount of corresponding anticodon tRNA expressed.

As used herein, the term “wild-type R. reniformis GFP” refers to the nucleic acid of SEQ ID NO: 5.

As used herein, the term “increased fluorescence intensity” or “increased brightness” refers to fluorescence intensity or brightness that is greater than that exhibited by wild-type R. reniformis GFP under a given set of conditions. Generally, an increase in fluorescence intensity or brightness means that fluorescence of a variant is at least 5% or more, and preferably 10%, 20%, 50%, 75%, 100% or more, up to even 5 times, 10 times, 20 times, 50 times or 100 times or more intense or bright than wild-type R. reniformis GFP under a given set of conditions.

As used herein, “recombinant polynucleotide” refers to a DNA sequence of two or more distinct nucleic acid sequences linked so as to encode a “humanized” Renilla reniformis green fluorescent protein (hrGFP), or a variant thereof, that has a heterologous amino acid sequence inserted internally into hrGFP, such that the hrGFP serves as a scaffold for presentation of the “heterologous” peptide. As used herein, “heterologous” nucleic acid sequence or amino acid sequence means an additional amino acid sequence or nucleic acid sequence that is not normally present in hrGFP. The “heterologous peptide” sequence can be as small as 2 amino acids, up to 50 amino acids. The “heterologous” sequence can be a nucleic acid sequence that contains at least one, preferably more than one, restriction enzyme cleavage or restriction site/s, thus creating a “cloning site” or “multiple cloning site”. The “multiple cloning site” contains restriction enzyme cleavage or recognition site/s, wherein an additional “heterologous nucleic sequence” can be inserted in such a manner that the sequence is in frame to the hrGFP coding sequence. The “heterologous” sequence can also be fused in frame with hrGFP via linkers. A “heterologous” nucleic acid sequence or amino acid sequence can be a known sequence of interest or a random sequence.

As used herein, “random peptide” and “random nucleic acid” refer to sequences that consist of random amino acids or nucleotides, respectively. Random peptide or nucleic acid molecules are not synthesized using a template of known sequence. That is, random nucleic acids can be synthesized by the incorporation of any nucleotide, at any position throughout the sequence. Thus, random nucleotide sequences can encode random peptides that contain randomly placed amino acids throughout the peptide. “Randomized peptide libraries” can be generated in the synthetic process by allowing the formation of all, or most of all, possible nucleotide position combinations throughout the nucleic acid. For example, a random oligonucleotide of 24 nucleotides would encode more than 10 billion eight amino acid peptides. Libraries typically range in size from 103 to 10⁹ different species, thus sub-sets of libraries may be made. As used herein, a “random peptide library”, also includes biased libraries. In a “biased” library, for example, particular amino acid residues are fixed while other residues vary at random, within a peptide sequence. Residues may be fixed such that there is structural bias. For example, the presence of cysteines to allow for disulfide bonds, prolines to create SH₃ domains, dimerization sequences, or amino acids that can be phosphorylated to generate protein-protein interaction sites. Several examples of suitable biases are described in U.S. application 2001/0003650, and are hereby incorporated by reference.

Random, biased, or known heterologous nucleotide sequences can be generated in a variety of ways. Such sequences can be generated, for example by oligonucleotide synthesis, or by PCR amplification from natural nucleic sequences, such as mRNA or genomic DNA. As used, herein, a “library of recombinant peptides” has diversity of randomized expression products ranging from at least 10³, and preferably 10⁷, 10⁸, or 10⁹ or more individual species. A “library of recombinant vectors” has diversity of randomized recombinant polynuceotide hrGFP encoding sequences that encode randomized expression products ranging from at least 10³, and preferably 10⁷, 10⁸, or 10⁹ or more individual species.

As used herein, “vector” refers to a DNA or RNA molecule that can replicate in a given host cell. A “recombinant vector”, is a vector that contains an inserted foreign nucleic acid sequence. A vector can be introduced into a host cell by a variety of means known to those skilled in the art, including, for example, transfection, electroporation, infection etc. When a “recombinant vector” is introduced into a host cell, it can transiently or stably present the foreign nucleic acid.

As used herein, a “host cell” refers to a cell of eukaryotic, prokaryotic, or archebacterial origin wherein a vector can be introduced. Examples of host cells include, but are not limited to Drosophila melangaster cells and other insect cells, Saccharomyces cerevisiae and other fungal cells, E. coli, Bacillus subtilis and other bacterial cells, as well as mammalian cells including immortalized cell lines and cells isolated from human tissues and cancers. A “host cell” can be additionally engineered to contain exogenous nucleic acid other than that provided by the recombinant vector that presents the recombinant polynucleotide encoding hrGFP.

As used herein, a “plurality of cells” is a population of cells preferably, but not necessarily of same type or strain. As used herein, a library can be introduced into a “plurality of cells”, generally from about 10³ to 10⁹ cells, such that each tranduced cell contains a recombinant vector that encodes a recombinant hrGFP polypeptide. When retroviral infection is used to introduce a recombinant polypeptide library, each infected cell will contain an individual species of recombinant hrGFP polypeptide. When other methods for introduction are used, the number of recombinant polypeptide species within a given cell can vary widely.

As used herein, peptide libraries are screened to identify peptides that confer a “phenotype of interest”. A “phenotype of interest” is a detectably altered phenotype relative to a wild-type or known starting phenotype, wherein the alteration represents a desired change in said wild-type or starting phenotype. “detectably altered” means at least a 10% change in the phenotype characteristic being measured.

“Phenotypes of interest” include, but are not limited to, morphological changes such as membrane ruffle, changes in cell growth, cell viability, cell-cell adhesion, or cell density, as well as changes in cellular transport of molecules within, or outside of a cell, and changes in membrane potential. A “phenotype of interest” may be a change in expression, the half-life, the location, or specific activity of, RNA, protein, lipids, hormones, signal transduction molecules, cytokines, and other molecules. “Phenotypes of interest” also include changes in susceptibility of a cell to infection by a pathogen, whether viral, bacterial, fungal, or any other. In one embodiment the “phenotype of interest” is an interaction of a peptide with a target molecule, DNA, RNA, or protein. For example, the peptide library described herein can be screened in yeast or mammalian two-hybrid and three hybrid systems, wherein the “phenotype of interest” is a change in the expression of a reporter molecule that indicates a peptide interaction.

The “phenotypes of interest” can be detected by any means known in the art and the assay will depend upon the phenotype to be measured. For example, membrane potentials can be monitored by patch-clamp techniques, morphological changes by microscopic analysis, changes in expression by western, northern, Southern, PCR, immunohistochemistry, or FACS analysis, etc. Susceptibility of cells to pathogens may be monitored by cell viability assays, syncytial assays, or any other standard assay used in the art. Reporter molecules, vectors, and systems can be used to assay for a particular phenotype. In addition, reporter cells can be used—for example, a second cell may respond to a signal provided by a first cell exhibiting the phenotype of interest.

As used herein, “inserted internally” or “fused internally” means that a heterologous DNA sequence is placed within the DNA sequence that encodes “humanized” Renilla reniformis green fluorescent protein (hrGFP), such that the heterologous sequence is linked in frame with, and flanked by, hrGFP encoding nucleotides. A heterologous DNA sequence, encoding a heterologous peptide that is “inserted internally” is linked to DNA that encodes hrGFP in such a manner that when the full length DNA is expressed, a recombinant hrGFP is generated that scaffolds the heterologous peptide. The heterologous peptide is “fused internally” into hrGFP. The heterologous peptides are “fused internally” such that hrGFP retains its autofluorescence and the hrGFP recombinant polypeptide has at least 1% of wild-type fluorescence, preferably 10% of wild-type fluorescence, more preferably 50-60%, and most preferably 95-100% of wild-type fluorescence. The recombinant hrGFP polypeptide can also have increased fluorescence intensity relative to wild-type (e.g. 100%, 120%, etc.).

As used herein, “recombinant polypeptide” refers to a heterologous amino acid sequence of two or more amino acids fused in frame to R. reniformis GFP or a variant thereof. One fused heterologous domain is inserted internally or linked to the N or C termini of the R. reniformis GFP polypeptide or variant thereof. Additional, fused heterologous domains may be inserted internally or linked to the N or C termini of the R. reniformis GFP polypeptide or variant thereof.

As used herein, the term “fused to the amino-terminal end” refers to the linkage of a polypeptide sequence to the amino terminus of another polypeptide. The linkage may be direct or may be mediated by a short (e.g., about 2-20 amino acids) linker peptide. Examples of useful linker peptides include, but are not limited to, glycine polymers ((G)ₙ) including glycine-serine and glycine-alanine polymers. It should be understood that the amino-terminal end as used herein refers to the existing amino-terminal amino acid of a polypeptide, whether or not that amino acid is the amino termal amino acid of the wild type or a variant form (e.g., an amino-terminal truncated form) of a given polypeptide.

As used herein, the term “fused to the carboxy-terminal end” refers to the linkage of a polypeptide sequence to the carboxyl terminus of another polypeptide. The linkage may be direct or may be mediated by a linker peptide. As with fusion to the amino-terminal end, fusion to the carboxy-terminal end refers to linkage to the existing carboxy-terminal of a polypeptide.

As used herein, the term “linker sequence” refers to a short (e.g., about 1-20 amino acids) sequence of amino acids that is not part of the sequence of either of two polypeptides being joined. A linker sequence is attached on its amino-terminal end to one polypeptide or polypeptide domain and on its carboxyl-terminal end to another polypeptide or polypeptide domain.

As used herein, the term “excitation spectrum” refers to the wavelength or wavelengths of light that, when absorbed by a fluorescent polypeptide molecule of the invention, causes fluorescent emission by that molecule.

As used herein, the term “emission spectrum” refers to the wavelength or wavelengths of light emitted by a fluorescent polypeptide.

As used herein, the term “operably linked” means that a given coding sequence is joined to a given transcriptional regulatory sequence such that transcription of the coding sequence occurs and is regulated by the regulatory sequence. Herein, a reporter gene is “functionally linked” to a DNA sequence for a DNA binding domain fusion protein such that the DNA binding domain fusion protein, which contains a peptide of interest, binds to the DNA sequence allowing for display of the peptide of interest. To be “functionally linked” the expression of the reporter gene can be regulated by a transactivation domain fusion protein, wherein the transactivation domain fusion protein contains a random or nonrandom peptide sequence that, upon interaction with a displayed peptide of interest, permits the transactivation of transcription of the reporter gene.

As used herein, the term “reporter construct” refers to a polynucleotide construct encoding a detectable reporter gene, linked to a transcriptional regulatory sequence conferring regulated transcription upon the polynucleotide encoding the detectable molecule.

As used herein, the terms, “transactivation protein” or “transactivation domain” refers to a protein or domain of a protein which can increase the transcription of a gene through interactions with the enzymes and factors that assemble at the promoter of a gene to form a functional transcription complex relative to transcription in the absence of active transactivating protein or domain. A transactivating protein or transactivation domain can exist in an active form, capable of effecting an increase in transcription, or, in an inactive form requiring activation before effecting an increase in transcription; a transactivating protein or transactivation domain of this type is referred to herein as “conditionally active”. It should be understood that a transactivating protein or transactivation domain can confer transactivating properties upon another protein or protein domain when expressed as a fusion with, or when bound to, that protein or protein domain. As used in the invention, a transactivation domain does not have sequence-specific DNA binding ability.

As used herein, the term “conditionally active” refers to a protein or domain of a protein which can exist in an active functional form or in an inactive form. This conditional activity can be regulated, for example, by phosphorylation, conformational change, or by complex formation with another protein. It should be understood that a conditionally active functional domain can confer conditional functional properties upon another protein or protein domain when expressed as a fusion with that protein or protein domain.

I. How to Make Humanized Recombinant R. reniformis GFP Polynucleotides and Polypeptides According to the Invention.

A number of methodologies are useful to provide the invention disclosed herein, including molecular, cellular and biochemical approaches. Polynucleotides encoding R. reniformis GFP are obtained in any of several different ways, including direct chemical synthesis, library screening and PCR amplification. R. reniformis GFP polypeptides are obtained by expression from recombinant polynucleotide sequences in appropriate organisms. Humanized R. reniformis GFP polypeptides and variants thereof are produced in similar ways following the introduction of mutations to the polynucleotide sequence encoding wild-type R. reniformis GFP. Those methodologies necessary to make and use the R. reniformis GFP polynucleotides, polypeptides and variants thereof of the invention are discussed in detail below.

A. Isolation of R. reniformis GFP-Encoding Polynucleotide Sequences.

1. R. reniformis cDNA Library Preparation.

Construction methods for libraries in a variety of different vectors, including, for example, bacteriophage, plasmids, and viruses capable of infecting eukaryotic cells are well known in the art. Any known library production method resulting in largely full-length clones of expressed genes may be used to provide a template for the isolation of GFP-encoding polynucleotides from R. reniformis.

For the library used to isolate the GFP-encoding polynucleotides disclosed herein, the following method was used. Poly(A) RNA was prepared from R. reniformis organisms as described by Chomczynski, P. and Sacchi, N. (1987, Anal. Biochem. 162: 156-159). cDNA was prepared using the ZAP-cDNA Synthesis Kit (Stratagene cat.# 200400) according to the manufacturer's recommended protocols, and inserted between the EcoR I and Xho I sites in the vector Lambda ZAP II. The resulting library contained 5×10⁶ individual primary clones, with an insert size range of 0.5-3.0 kb and an average insert size of 1.2 kb. The library was amplified once prior to use as template for PCR reactions.

2. Isolation of R. reniformis GFP Coding Sequence by PCR.

The R. reniformis GFP coding sequence was isolated by polymerase chain reaction (PCR) amplification of the sequence from within the cDNA library described herein. A large number of PCR methods are known to those skilled in the art. Thermal-cycled PCR (Mullis and Faloona, 1987, Methods Enzymol., 155: 335-350; see also, PCR Protocols, 1990, Academic Press, San Diego, Calif., USA for a review of PCR methods) uses multiple cycles of DNA replication catalyzed by a thermostable, DNA-dependent DNA polymerase to amplify the target sequence of interest. Briefly, oligonucleotide primers are selected such that they anneal on either side and on opposite strands of a sequence to be amplified. The primers are annealed and extended using a template-dependent thermostable DNA polymerase, followed by thermal denaturation and annealing of primers to both the original template sequence and the newly-extended template sequences, after which primer extension is performed. Repeating such cycles results in exponential amplification of the sequences between the two primers.

In addition to thermal cycled PCR, there are a number of other nucleic acid sequence amplification methods that can be used to amplify and isolate a GFP-encoding polynucleotide according to the invention from an R. reniformis cDNA library. These include, for example, isothermal 3SR (Gingeras et al., 1990, Annales de Biologie Clinique, 48(7): 498-501; Guatelli et al., 1990, Proc. Natl. Acad. Sci. U.S.A., 87: 1874), and the DNA ligase amplification reaction (LAR), which permits the exponential increase of specific short sequences through the activities of any one of several bacterial DNA ligases (Wu and Wallace, 1989, Genomics, 4: 560). The contents of both of these references are incorporated herein in their entirety by reference.

To amplify a sequence encoding R. reniformis GFP from an R. reniformis cDNA library, the following approach was taken. The R. reniformis GFP coding sequence was amplified using the 5′ primer 5′-AATTATTAGAATTCACCATGGTGAGTAAACAAATATTGAAGAAC-3′ (SEQ ID NO: 6) and the 3′ primer 5′-ATAATATTCTCGAGTTAAACCCATTCGTGTAAGGATCC-3 (SEQ ID NO: 7). The 5′ primer contains an EcoR I recognition site to facilitate subsequent cloning of the amplified fragment, followed by the Kozak consensus translation initiation sequence ACCATGG. The 3′ primer contains an Xho I recognition site to facilitate cloning of the amplified fragment. Oligonucleotides may be purchased from any of a number of commercial suppliers (for example, Life Technologies, Inc., Operon Technologies, etc.). Alternatively, oligonucleotide primers may be synthesized using methods well known in the art, including, for example, the phosphotriester (see Narang, S. A., et al., 1979, Meth. Enzymol., 68: 90; and U.S. Pat. No. 4,356,270), phosphodiester (Brown, et al., 1979, Meth. Enzymol., 68: 109), and phosphoramidite (Beaucage, 1993, Meth. Mol. Biol., 20: 33) approaches. Each of these references is incorporated herein in its entirety by reference.

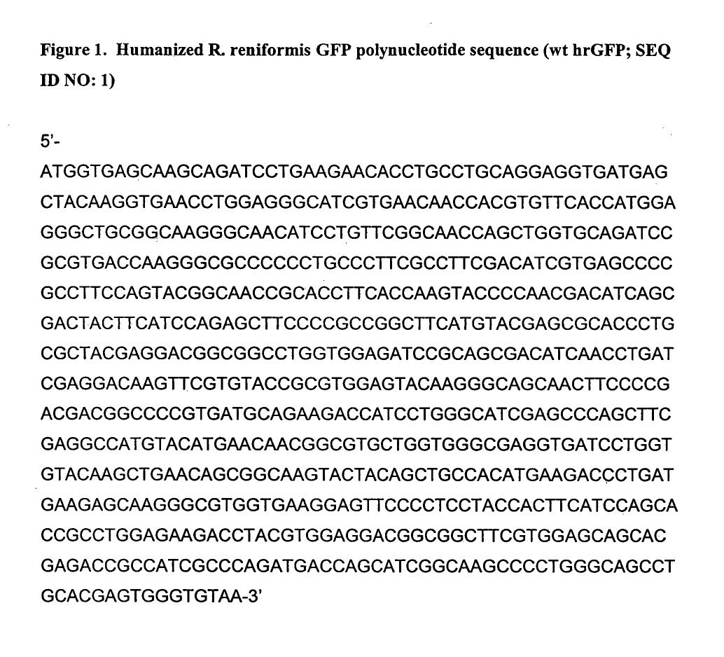

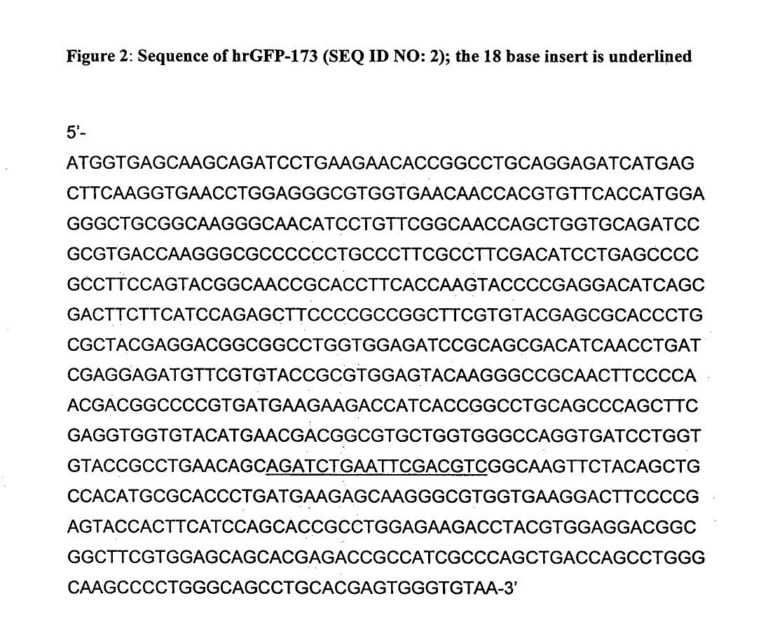

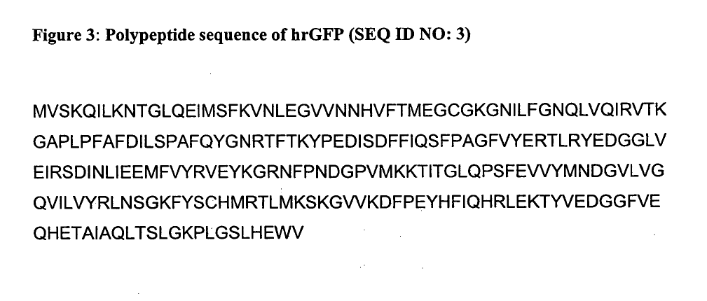

PCR was carried out in a 50 μl reaction volume containing 1× TaqPlus Precision buffer (Stratagene), 250 μM of each dNTP, 200 nM of each PCR primer, 2.5 U TaqPlus Precision enzyme (Stratagene) and approximately 3×10⁷ lambda phage particles from the amplified cDNA library described above. Reactions were carried out in a Robocycler Gradient 40 (Stratagene) as follows: 1 min at 95° C. (1 cycle), 1 min at 95° C., 1 min at 53° C., 1 min at 72° C. (40 cycles), and 1 min at 72° C. (1 cycle). Reaction products were resolved on a 1% agarose gel, and a band of approximately 700 bp was excised and purified using the StrataPrep DNA Gel Extraction Kit (Stratagene). Other methods of isolating and purifying amplified nucleic acid fragments are well known to those skilled in the art. The PCR fragment was subcloned by digestion to completion with EcoRI and XhoI and insertion into the retroviral expression vector pFB (Stratagene) to create the vector pFB-rGFP. Both strands of the cloned GFP fragment were completely sequenced. The coding polynucleotide and amino acid sequences are presented in FIGS. 1 and 2, respectively. The R. reniformis and R. mulleri GFP coding sequences are 83% homologous, and the proteins share 88% identical amino acid sequence.

3. Isolation of R. reniformis GFP-Encoding Polynucleotides by Library Screening.

An alternative method of isolating GFP-encoding polynucleotides according to the invention involves the screening of an expression library, such as a lambda phage expression library, for clones exhibiting fluorescence within the emission spectrum of GFP when illuminated with light within the excitation spectrum of GFP. In this way clones may be directly identified from within a large pool. Standard methods for plating lambda phage expression libraries and inducing expression of polypeptides encoded by the inserts are well established in the art. Screening by fluorescence excitation and emission is carried out as described herein below using either a spectrofluorometer or even visual identification of fluorescing plaques. With either method, fluorescent plaques are picked and used to re-infect fresh cultures one or more times to provide pure cultures, from which GFP insert sequences may be determined and sub-cloned.

As another alternative, if a sequence is available for the polynucleotide one wishes to obtain, the polynucleotide may be chemically synthesized by one of skill in the art. The same synthetic methods used for the preparation of oligonucleotide primers (described above) may be used to synthesize gene coding sequences for GFPs of the invention. Generally this would be performed by synthesizing several shorter sequences (about 100 nt or less), followed by annealing and ligation to produce the full length coding sequence.

B. Generation of Humanized R. reniformis GFP-Encoding Polynucleotide Sequences.

Herein, the nucleic acid sequence of wild-type R. reniformis GFP is modified to enhance its expression in mammalian or human cells. The codon usage of R. reniformis is optimal for expression in R. reniformis, but not for expression in mammalian or human systems. Therefore, the adaptation of the sequence isolated from the sea pansy for expression in higher eukaryotes involves the modification of specific codons to change those less favored in mammalian or human systems to those more commonly used in these systems. This so-called “humanization” is accomplished by site-directed mutagenesis of the less favored codons as described herein or as known in the art. Similar modifications of the A. victoria GFP coding sequences are described in U.S. Pat. No. 5,874,304. The preferred codons for human gene expression are listed in Table 1. The codons in the table are arranged from left to right in descending order of relative use in human genes. Consideration of the codons in wild-type R. reniformis GFP (for example, SEQ ID NO: 5) relative to those favored in human genes allows one of skill in the art to identify which codons to modify in the R. reniformis GFP gene to achieve more efficient expression in human or mammalian cells. In particular, those codons underlined in the table are used in less than ten per one thousand codons in known human genes and, if found in the R. reniformis sequence would therefore represent the most important codons to modify for enhanced expression efficiency in mammalian or human cells.

TABLE 1
PREFERRED DNA CODONS FOR HUMAN USE

| | |
|---|---|
| Amino Acids | Codons Preferred in Human Genes |

| | | | |
|---|---|---|---|
| Alanine | Ala | A | GCC GCT GCA GCG |
| Cysteine | Cys | C | TGC TGT |
| Aspartic acid | Asp | D | GAC GAT |
| Glutamic acid | Glu | E | GAG GAA |
| Phenylalanine | Phe | F | TTC TTT |
| Glycine | Gly | G | GGC GGG GGA GGT |
| Histidine | His | H | CAC CAT |
| Isoleucine | Ile | I | ATC ATT ATA |
| Lysine | Lys | K | AAG AAA |
| Leucine | Leu | L | CTG TTG CTT CTA TTA |
| Methionine | Met | M | ATG |
| Asparagine | Asn | N | AAC AAT |
| Proline | Pro | P | CCC CCT CCA CCG |
| Glutamine | Gln | Q | CAG CAA |
| Arginine | Arg | R | CGC AGG CGG AGA CGA CGT |
| Serine | Ser | S | AGC TCC TCT AGT TCA TCG |
| Threonine | Thr | T | ACC ACA ACT ACG |
| Valine | Val | V | GTG GTC GTT GTA |
| Tryprophan | Trp | W | TGG |
| Tyrosine | Tyr | Y | TAC TAT |

The codons at the left represent those most preferred for use in human genes, with human usage decreasing towards the right. Underlined codons are used in less than 10 per 1000 codons used in human genes.

A humanized version of R. reniformis GFP has been generated and is represented by SEQ ID NO: 1.

C. Variants of Humanized R. reniformis GFP According to the Invention.

Herein, a humanized R. reniformis GFP (hrGFP) nucleic acid is modified by the insertion of a heterologous nucleic acid sequence into the coding sequence of hrGFP. The heterologous sequence can be a random or specific sequence, for example a known multiple cloning site sequence. Herein, a multiple cloning site sequence has been inserted between nucleotides 519 and 520 of hrGFP (SEQ ID NO: 2) using methods known in the art (see Example 1). The recombinant polynucleotide encodes a recombinant polypeptide that retains its autofluoresence. Thus, the recombinant polynucleotide of SEQ ID NO: 2 is an example of a nucleotide sequence, wherein an additional nucleic acid heterologous sequences can be inserted in frame with hrGFP. It should be understood that the present invention also encompasses insertions within other regions of the humanized R. reniformis GFP. For example, one skilled in the art can readily determine whether hrGFP comprising heterologous in frame insertions retain autofluorescence by expressing such proteins (e.g. or λ phage) and irradiating the proteins or cells expressing them with light in the excitation spectrum of hrGFP and measuring emitted fluoresence.

One way to identify other sites for the insertion of heterologous sequence is to insert the multiple cloning sequence described herein (or another multiple cloning sequence) at in-frame insertions of 3 nucleotides, or multiples thereof into the nucleic acid sequence. For example, a multiple cloning site could be inserted in-frame into SEQ ID NO. 1 between amino acid coding nucleotides 3 and 4, 6 and 7, 9 and 10, 12 and 13, etc., e.g., between amino acid coding nucleotides 75 and 76, 90 and 91, 120 and 121, 150 and 151, 173 and 174, 180 and 181, etc. Measurement of fluorescence for such clones will determine which insertion sites are tolerated by the hrGFP protein. The fluorescence retained by the insertion mutant should be at least 1% that of wild-type hrGFP, preferably at least 10%, more preferably at least 50%, 60%, 70% or more, most preferably 90%, 95%, 98%, 99% or more, including 100% or more. It should be understood that such insertions may change the excitation or emission spectra of the hrGFP polypeptide, but it is within the ability of one of ordinary skill in the art to scan a given polypeptide with various excitation energies and detect varied emission spectra.

Alternatively, specific sites can be selected for insertion based on the characterization of the hrGFP polypeptide by, e.g. crystallography, NMR or CD, which will identify solvent exposed region of the polypeptide which are more likely to tolerate such insertion while retaining fluorescence.

The use of a hrGFP vector that contains a multiple cloning site within the coding sequence of hrGFP is desirable, for it permits efficient ligation of random nucleic acid sequences for the generation of random peptide libraries wherein hrGFP is a scaffold.

Generation of Random Heterologous Sequences

In one embodiment a random peptide GFP scaffolded library is generated. In a preferred embodiment, a hrGFP vector contains a multiple cloning site within the coding sequence of hrGFP. The multiple cloning site is used to insert at least one randomized nucleic acid sequence in frame with hrGFP. The randomized sequence is inserted such that the encoded random peptide is displayed in solvent exposed regions of the GFP protein. The random peptide libraries can be generated by synthetic processes known in the art, allowing the formation of all, or essentially all, possible nucleotide position combinations throughout the randomized nucleic acid sequence. One manner in which the library can be generated is by synthetic oligonucleotide sysnthesis. Alternatively, the library can be generated from genomic DNA or mRNA from a natural source, in which case appropriate restriction sites are added by PCR during amplification for easy in frame ligation of peptide sequences. The Generated DNA library sequences are inserted into the appropriate hrGFP expression vector by standard molecular biology techniques. A variety of suitable expression vectors are described herein.

Herein, a randomized peptide library, also includes biased libraries. For example, individual amino acid residues are fixed within a randomized peptide sequence. Residues can be fixed such that there is structural bias. Residues that can be fixed within an otherwise randomized sequence include, for example, cysteines to allow for disulfide bonds, prolines to create SH₃ domains, dimerization sequences, or amino acids that can be phosphorylated to generate protein-protein interaction sites. Several examples of suitable biases are described in U.S. application 2001/0003650, and are hereby incorporated by reference.

The library of recombinant vectors useful according to the invention should have diversity of randomized recombinant polynuceotide hrGFP encoding sequences that encode randomized expression products ranging from at least 10³, and preferably to 10⁷, 10⁸, 10⁹ or more individual species.

The invention further provides for the insertion of peptides into hrGFP using linker sequences. The linkage can be mediated by a short (e.g., about 2-20 amino acids) linker peptide. Examples of useful linker peptides include, but are not limited to, glycine polymers ((G)ₙ) including glycine-serine and glycine-alanine polymers. The linker essentially tethers the peptide sequence to hrGFP, permitting greater exposure or more flexible presentation of the inserted peptide sequence. Suitable linker sequences are apparent to those skilled in the art.

Variants with Increased Brightness

Humanized R. reniformis GFP variants with increased brightness relative to wild-type R. reinformis GFP, and other modifications are also of interest. For example, variants exhibiting shifts in either excitation or emission spectra or both are useful since they allow the monitoring of the location or level of more than one polypeptide in the same cell through simple fluorescence measurements. Also, GFP variants with, for example, an excitation spectrum that is overlapped by the emission spectrum of another GFP can be useful for FRET-based assays. Alternatively, GFP variants whose spectral characteristics are responsive to environmental changes, such as pH or oxidation/reduction status or are responsive to changes in phosphorylation status are useful in studies of such intracellular or even extracellular changes.

a. Mutagenesis Methods Useful According to the Invention

Modifications to the R. reniformis GFP coding sequences can be either random or targeted. In either case, selection involves monitoring individual clones for the desired modified characteristic, be it enhanced fluorescence relative to wild-type R. reniformis GFP, a spectral shift, or other modification.

Many random and site-directed mutagenesis methods are known in the art, and any of them that generate modifications to the R. reniformis GFP coding sequence of SEQ ID NO: 1 are applicable to generate variant GFPs useful according to the invention. Several examples of both random and site-directed mutagenesis are described below.

Random Mutagenesis

Chemical mutagenesis using, for example, nitrous acid, permanganate or formic acid may be used to generate random mutations essentially as described by Meyer et al., 1985, Science 229: 242, which is incorporated herein in its entirety by reference. When following the Meyer et al. method, a mutated population of single-stranded R. reniformis GFP gene fragments is generated that is then amplified using the PCR primers used herein above for amplification of wild-type R. reniformis GFP. The amplification products, bearing random mutations, are cloned into an appropriate vector and transformed into bacteria. Colonies are screened for altered fluorescence characteristics relative to wild-type R. reniformis GFP either expressed from the same vector in the same bacterial strain or purified.

An alternative to chemical mutagenesis for the generation of random mutants is the use of a mutagenic bacterial strain, such as the XL1-Red E. coli strain (Stratagene), which is deficient in DNA polymerase proofreading activity and DNA repair machinery. A plasmid introduced to this or a similar strain of bacteria becomes mutated during cell division. When using a mutagenic bacterial strain such as XL1-Red, plasmids containing the GFP sequence to be mutagenized (i.e., SEQ ID NO: 1) are transformed into the mutagenic bacteria and propagated for about two days (shorter or longer, depending upon the desired degree of mutagenesis). The randomly mutated plasmids are isolated from the culture using standard methods and re-transformed into non-mutagenic bacteria (e.g., E. coli strain DH5α; Life Technologies, Inc.), which are plated to achieve individual colonies. The colonies are then screened for the desired altered fluorescence characteristic relative to colonies expressing wild-type R. reniformis from the same plasmid in the same bacterial strain.

Another example of a method for random mutagenesis is the so-called “error-prone PCR method”. As the name implies, the method amplifies a given sequence under conditions in which the DNA polymerase does not support high fidelity incorporation. The conditions encouraging error-prone incorporation for different DNA polymerases vary, however one skilled in the art may determine such conditions for a given enzyme. A key variable for many DNA polymerases in the fidelity of amplification is, for example, the type and concentration of divalent metal ion in the buffer. The use of manganese ion and/or variation of the magnesium or manganese ion concentration may therefore be applied to influence the error rate of the polymerase. As with the other methods, mutagenized sequences are inserted into an appropriate vector, transformed into bacteria and screened for the desired characteristics.

Site-Directed or Targeted Mutagenesis

There are a number of site-directed mutagenesis methods known in the art which allow one to mutate a particular site or region in a straightforward manner. These methods are embodied in a number of kits available commercially for the performance of site-directed mutagenesis, including both conventional and PCR-based methods. Examples include the EXSITE™ PCR-based site-directed mutagenesis kit available from Stratagene (Catalog No. 200502; PCR based) and the QUIKCHANGE™ site-directed mutagenesis kit from Stratagene (Catalog No. 200518; PCR based), and the CHAMELEON® double-stranded site-directed mutagenesis kit, also from Stratagene (Catalog No. 200509).

Older methods of site-directed mutagenesis known in the art relied upon sub-cloning of the sequence to be mutated into a vector, such as an M13 bacteriophage vector, that allows the isolation of single-stranded DNA template. In these methods one annealed a mutagenic primer (i.e., a primer capable of annealing to the site to be mutated but bearing one or more mismatched nucleotides at the site to be mutated) to the single-stranded template and then polymerized the complement of the template starting from the 3′ end of the mutagenic primer. The resulting duplexes were then transformed into host bacteria and plaques were screened for the desired mutation.

More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

The protocol described below accommodates these considerations through the following steps. First, the template concentration used is approximately 1000-fold higher than that used in conventional PCR reactions, allowing a reduction in the number of cycles from 25-30 down to 5-10 without dramatically reducing product yield. Second, the restriction endonuclease DpnI.

(recognition target sequence: 5-Gm6ATC-3, where the A residue is methylated) is used to select against parental DNA, since most common strains of E. coli Dam methylate their DNA at the sequence 5′-GATC-3′. Third, Taq Extender is used in the PCR mix in order to increase the proportion of long (i.e., full plasmid length) PCR products. Finally, Pfu DNA polymerase is used to polish the ends of the PCR product prior to intramolecular ligation using T4 DNA ligase.

The method is described in detail as follows:

PCR-Based Site Directed Mutagenesis

Plasmid template DNA (approximately 0.5 pmole) is added to a PCR cocktail containing: 1× mutagenesis buffer (20 mM Tris HCl, pH 7.5; 8 mM MgCl₂; 40 ug/ml BSA); 12-20 pmole of each primer (one of skill in the art may design a mutagenic primer as necessary, giving consideration to those factors such as base composition, primer length and intended buffer salt concentrations that affect the annealing characteristics of oligonucleotide primers; one primer must contain the desired mutation, and one (the same or the other) must contain a 5′ phosphate to facilitate later ligation), 250 uM each dNTP, 2.5 U Taq DNA polymerase, and 2.5 U of Taq Extender (Available from Stratagene; See Nielson et al. (1994) Strategies 7: 27, and U.S. Pat. No. 5,556,772). The PCR cycling is performed as follows: 1 cycle of 4 min at 94° C., 2 min at 50° C. and 2 min at 72° C.; followed by 5-10 cycles of 1 min at 94° C., 2 min at 54° C. and 1 min at 72° C. The parental template DNA and the linear, PCR-generated DNA incorporating the mutagenic primer are treated with DpnI (10 U) and Pfu DNA polymerase (2.5 U). This results in the DpnI digestion of the in vivo methylated parental template and hybrid DNA and the removal, by Pfu DNA polymerase, of the non-template-directed Taq DNA polymerase-extended base(s) on the linear PCR product. The reaction is incubated at 37° C. for 30 min and then transferred to 72° C. for an additional 30 min. Mutagenesis buffer (115 ul of 1×) containing 0.5 mM ATP is added to the DpnI-digested, Pfu DNA polymerase-polished PCR products. The solution is mixed and 10 ul are removed to a new microfuge tube and T4 DNA ligase (2-4 U) is added. The ligation is incubated for greater than 60 min at 37° C. Finally, the treated solution is transformed into competent E. coli according to standard methods.

Limited Random Mutagenesis

A subcategory of site-directed mutagenesis involves the use of randomized oligonucleotides to introduce random mutations into a limited region of a given sequence (this will be referred to as “limited random mutagenesis”). This is particularly useful when one wishes to mutate every base within, for example, a region encoding a hexapeptide. Generally, the oligonucleotides used for this type of approach have a stretch of constant nucleotides exactly complementary to a region on either side of and immediately adjacent to the region to be mutated, linked by a randomized or partially randomized oligonucleotide sequence corresponding to the sequence to be mutated. One of the constant sequences flanking the mutagenic region should have a restriction site to facilitate the replacement of wild-type sequence with the mutagenized sequence following mutagenesis. Ideally, such a restriction site is naturally present adjacent to the region to be mutated, but one skilled in the art may also introduce restriction sites through silent mutations, without altering the coding sequence (see, for example, the list of restriction sites that may be introduced by silent mutagenesis in the New England Biolabs (NEB) catalog appendices, specifically at pages 282-283 of the 1998/1999 NEB catalog).

In the limited random mutagenesis method, mutagenic oligonucleotides as described above are used, along with a selected partner primer, and a wild type, or even previously mutated, recombinant R. reniformis GFP construct template (wild-type, or, alternatively, previously altered) to PCR amplify a pool of fragments, all randomly or semi-randomly mutated at the desired sites. The partner primer is selected so that it is either 5′ or 3′ of the mutagenized stretch of nucleotides, and should have either a naturally occurring restriction site or an engineered restriction site that does not alter GFP coding sequences, to permit the replacement of the wild-type with the mutated sequences. Conveniently, the partner primer can bind in the vector sequences immediately 5′ or 3′ of the GFP coding sequence. The amplified pool of mutated fragments is cleaved with the restriction enzymes recognizing the respective sites in the mutagenic and partner primers, and the pool is ligated into a similarly cleaved recombinant vector comprising the GFP coding sequences (either 5′ of or 3′ of the mutagenized site) not amplified during the mutagenic step, to generate a pool of full length GFP coding sequences randomly or semi-randomly mutated only over the selected stretch of nucleotides.

The mutations in the limited random mutagenesis approach are referred to as “random or semi-random” because the mutagenic sequences do not necessarily have to be completely random. One of skill in the art will recognize, for example, that it is possible to vary one, two, or all three nucleotides in a codon with different results as far as the range of possible changes to the peptide sequence encoded, from no change (often possible in the third or “wobble” nucleotide) to limited change (changes affecting the middle and or third nucleotide only) to completely random change (changes affecting all three nucleotides of the codon). Therefore, by maintaining some nucleotides constant within the mutagenized region and allowing others to vary (either over all four possible nucleotides or over one or more subsets of them), the characteristics of the mutagenized region can be controlled. Sequences mutagenized in such a manner would be “semi-randomly” mutagenized. Following the cloning of the mutated pool of R. reniformis GFP vectors using the limited random mutagenesis method, or its equivalent, the mutated pool is transformed into bacteria, expression is induced, and the clones are screened for the desired altered characteristic.

b. Purification of R. reniformis GFP or Variants Thereof.

If necessary, R. reniformis GFP is purified from R. reniformis organisms as described by Ward and Cormier (1979, J. Biol. Chem. 254: 781-788) and by Matthews et al. (1977, Biochemistry 16: 85-91), the contents of both of which are herein incorporated by reference. Similar procedures may be applied by one of skill in the art to bacterially expressed R. reniformis GFP or variants thereof following freeze-thaw lysis and preparation of a clarified lysate by centrifugation at 14,000×g. Briefly, the methods employed by Matthews et al. and Ward and Cormier involve successive chromatography over DEAE-cellulose, Sephadex G-100, and DTNB (5,5′-dithiobis(2-nitrobenzoic acid))-Sepharose columns, and dialysis against 1 mM Tris (pH 8.0), 0.1 mM EDTA. The dialyzed fractions containing GFP (identified by fluorescence) are then acid treated to precipitate contaminants, followed by neutralization of the supernatant, which is lyophilized. Low salt (10 mM to 1 mM initially) and pH ranging from 7.5 to 8.5 are critical to maintaining activity upon lyophilization. The lyophilized sample is re-suspended in water, immediately centrifuged to remove less-soluble contaminants and applied to a Sephadex G-75 column. GFP is eluted in 1.0 mM Tris (pH 8.0), 0.1 mM EDTA. Samples are concentrated by partial lyophilization and dialyzed against 5 mM sodium acetate, 5 mM imidazole, 1 mM EDTA, pH 7.5, followed by chromatography over a DEAE-BioGel-A column equilibrated in the same dialysis buffer. GFP is eluted with a continuous acidic gradient from pH 6.0 to 4.9 in the same acetate/imidizole buffer. Following dialysis of GFP-containing fractions against 1.0 mM Tris-HCl, 0.1 mM EDTA, pH 8.0, the sample is partially lyophilized to concentrate and passed over a Sephadex G-75 (Superfine) column. The GFP-containing fractions are then loaded onto a DEAE-BioGel A column in Tris/EDTA buffer at pH 8.0, followed by elution in a continuous alkaline gradient from pH 8.5 to 10.5 formed with 20 mM glycine, 5 mM Tris-HCl and 5 mM EDTA. GFP-containing fractions contain essentially homogeneous R. reniformis GFP.

In screening applications requiring less pure GFP preparations, recombinant R. reniformis or variants thereof can be purified from bacteria as follows. Bacteria transformed with a recombinant GFP-encoding vector of the invention are grown in Luria-Bertani medium containing the appropriate selective antibiotic (e.g., ampicillin at 50 μg/ml). If the vector permits, recombinant polypeptide expression is induced by the addition of the appropriate inducer (e.g., IPTG at 1 mM). Bacteria are harvested by centrifugation and lysed by freeze-thaw of the cell pellet. Debris is removed by centrifugation at 14,000×g, and the supernatant is loaded onto a Sephadex G-75 (Pharmacia, Piscataway, N.J.) column equilibrated with 10 mM phosphate buffered saline, pH 7.0. Fractions containing GFP are identified by fluorescence emission at 506 nm when excited by 500 nm light, or by excitation and emission over a range of spectra when purifying GFP variants with altered spectral characteristics.

c. Modifications to Humanized R. reniformis GFP Useful According to the Invention.

The R. reniformis chromophoric center is comprised of amino acids 64-69 of the wild-type polypeptide, which has the sequence FQYGNR (SEQ ID NO: 8). Mutation of this amino acid sequence at one or more positions, using for example, standard site-directed or limited random mutagenesis or its equivalent, can give rise to R. reniformis variants exhibiting enhanced fluorescence intensity or shifted spectral characteristics. Changes at sites outside of the chromophoric center can also affect the fluorescence properties of the polypeptide. For example, because R. reniformis lives at a temperature significantly below 37° C., mutations that stabilize the folded fluorescent form of the polypeptide at 37° C. may enhance the fluorescence of the polypeptide in human or mammalian cell culture, or in bacterial cultures, for that matter. Further, while the chemical nature of the R. reniformis GFP chromophore is nearly identical to that of the A. victoria GFP chromophore (Ward et al., 1980, Photochem. Photobiol. 31: 611-615), the fluorescence characteristics, including intensity and spectra are quite different. This indicates that modifications outside of the chromophoric center will likely have an impact on fluorescence characteristics.

D. Screening for R. reniformis GFP Mutants with Altered Fluorescence Characteristics or Altered Traits.

One method of screening for altered fluorescence characteristics involves lifting single bacterial colonies transformed with a mutated GFP sequence from a plate onto a support, such as 0.45 μm pore size nitrocellulose membranes (Schleicher & Schuell, Keene, N.H.), placing the membranes onto fresh agar/medium plates (e.g., LB agar containing 50 μg/ml ampicillin, 1 mM IPTG for a vector containing amp^{r} and lacI repressor genes, and a lac operator upstream of the R. reniformis GFP coding region), bacteria-side up, and allowing colonies to grow on the membrane. The membranes are then scanned for fluorescence characteristics of the colonies. Scanning can be performed under illumination with monochromatic light, for example as generated by passing light from a 150 W Xenon lamp (Xenon Corp., Woburn, Mass.) through interference filters appropriate for the desired excitation wavelengths (filters available, for example, from CVI Laser Corp., Albuquerque, N. Mex.). Emissions from the illuminated colonies may be observed through, for example, a Schott KV500 filter, which has a 500 nm wavelength cutoff. The same methods of screening mutants for altered fluorescence characteristics are applicable regardless of whether mutagenesis is random or targeted.

Alternative fluorescence scanning equipment includes a scanning polychromatic light source (such as a fast monochromator from T.I.L.L. Photonics, Munich, Germany) and an integrating RGB color camera (such as the Photonic Science Color Cool View). Following multi-wavelength excitation scanning, images captured by the integrating color camera may be subjected to image analysis to determine the actual color of the emitted light using software such as Spec R4 (Signal Analytics Corp., Vienna, Va., USA).

With many of the altered characteristics (e.g., fluorescence intensity, thermal stability or spectral characteristics) being screened for, bacteria or eukaryotic (e.g., yeast or mammalian) cells expressing the mutated form can first be screened relative to control cells expressing the wild-type form, followed if necessary by characterization of either clarified lysates or purified polypeptides from those colonies selected by the cellular screen. For other altered characteristics (e.g., pH sensitivity or phosphorylation-dependent alteration of fluorescence), purified polypeptides or at least clarified bacterial or eukaryotic cell lysates may be necessary for screening. Where necessary, clarified lysate preparation and/or purification is/are achieved according to methods described herein or known in the art. Ultimately, purified mutated or altered GFP polypeptides can be compared to wild-type R. reniformis GFP (native or recombinant) with regard to the characteristic one desires to modify. When screening for mutants of R. reniformis GFP with altered fluorescence intensity or brightness according to the invention, one looks for fluorescence that is at least two times more intense or bright than the fluorescence of wild-type R. reniformis GFP (either isolated from R. reniformis or expressed from a recombinant vector construct of the invention), and up to 3 times, 5 times, 10 times, 20 times, 50 times or even 100 or more times as intense or bright as the same molar amount of wild-type R. renifirmis GFP.

When screening for R. reniformis GFP mutants with altered spectral characteristics, one looks for GFP polypeptides that exhibit excitation or emission spectra that are distinguishable or detectably distinct from those of the wild-type GFP polypeptide. By distinguishable or detectably distinct is meant that standard filter sets allow either the excitation of one form without excitation of the other form, or similarly, that standard filter sets allow the distinction of the emission from one form from the other. Generally, distinguishable excitation or emission spectra have peaks that vary by more than 1 nm, and preferably vary by more than 2, 3, 4, 5, 10 or more nm. The peaks of distinguishable spectra are also preferably narrow, covering a range of about 5 nm or less, 7 nm or less, 10 nm or less, 15 nm or less, 20 nm or less, 50 nm or less, or 100 nm or less. The maximum allowable breadth of a peak that is considered distinguishable is directly related to how much the peak maximum varies from the maximum of the peak it is being distinguished from. In other words, the larger the variance between the peak wavelengths of two fluorescent polypeptides, the broader the peaks may be and still be distinguishable. Conversely, the lower the variance between the centers of the peaks, the narrower the peaks must be to be distinguishable.

Particularly preferred spectral shifts are shifts in emission spectra that are not accompanied by distinguishable shifts in excitation spectra. Such a shift permits the excitation of two or more different GFPs with light of the same wavelength (or same range of excitation wavelengths) yet also permits distinction of the fluorescence of two or more GFPs based on the different emission wavelengths.

Other preferred spectral shifts include those that render the R. reniformis GFP capable of FRET as either a donor or an acceptor fluoroprotein. For example, a spectral alteration that changes the excitation spectrum of a first fluorescent polypeptide so that it overlaps the emission spectrum of a second fluorescent polypeptide will define a pair of fluorescent polypeptides capable of FRET. It is preferred, although not necessary that both the first and second fluorescent polypeptides be GFP polypeptides; if a non-GFP fluorescent polypeptide is a donor or acceptor for FRET, it is preferred that a polynucleotide sequence for that fluorescent polypeptide is known.

If both fluorescent polypeptides of a FRET pair are R. reniformis GFP polypeptides, one or both polypeptides may be altered. That is, one may be wild-type R. reniformis GFP and the other may be altered, or both GFPs of the FRET pair may be altered. In the case in which wild-type R. reniformis GFP is a member of the pair, it may be either the donor or the acceptor member of the pair.

Another altered characteristic that may enhance the usefulness of the R. reniformis GFP polypeptides of the invention is altered stability of the polypeptide in vivo. As mentioned above, modifications that alter the folded stability of the polypeptide's fluorophore center can alter the fluorescence intensity of the polypeptide. However, modifications that increase or reduce the in vivo or in vitro half-life of the entire GFP polypeptide, i.e., modifications that affect polypeptide turnover or degradation are also useful. For example, increased stability can enhance the detection of the modified R. reniformis GFP by allowing a larger steady-state pool of GFP to accumulate at a given expression rate. Importantly, there is also usefulness for R. reniformis GFP polypeptide variants with reduced in vivo or in vitro stability. For example, the responsiveness of reporter assays for transcription is enhanced by reporter molecules with shorter half-lives. Generally, the shorter the biological half-life of the reporter molecule, the faster a new steady state is achieved when the transcription rate increases or decreases, enhancing the sensitivity of the assay.

E. Production of Humanized R. reniformis GFP Polypeptides and Variants Thereof.

The production of R. reniformis GFP polypeptides and variants thereof from recombinant vectors comprising GFP-encoding polynucleotides of the invention may be effected in a number of ways known to those skilled in the art. For example, plasmids, bacteriophage or viruses may be introduced to prokaryotic or eukaryotic cells by any of a number of ways known to those skilled in the art. Following introduction of R. reniformis GFP-encoding polynucleotides to a prokaryotic or eukaryotic cell, expressed GFP polypeptides may be isolated using methods known in the art or described herein below. Useful vectors, cells, methods of introducing vectors to cells and methods of detecting and isolating GFP polypeptides and variants thereof are also described herein below.

1. Vectors Useful According to the Invention.

There is a wide array of vectors known and available in the art that are useful for the expression of GFP polypeptides or variants thereof according to the invention. The selection of a particular vector clearly depends upon the intended use of the GFP polypeptide or variant thereof. For example, the selected vector must be capable of driving expression of the polypeptide in the desired cell type, whether that cell type be prokaryotic or eukaryotic. Many vectors comprise sequences allowing both prokaryotic vector replication and eukaryotic expression of operably linked gene sequences.

Vectors useful according to the invention may be autonomously replicating, that is, the vector, for example, a plasmid, exists extrachromosomally and its replication is not necessarily directly linked to the replication of the host cell's genome. Alternatively, the replication of the vector may be linked to the replication of the host's chromosomal DNA, for example, the vector may be integrated into the chromosome of the host cell as achieved by retroviral vectors.

Vectors useful according to the invention preferably comprise sequences operably linked to the GFP coding sequences that permit the transcription and translation of the GFP sequence. Sequences that permit the transcription of the linked GFP sequence include a promoter and optionally also include an enhancer element or elements permitting the strong expression of the linked sequences. The term “transcriptional regulatory sequences” refers to the combination of a promoter and any additional sequences conferring desired expression characteristics (e.g., high level expression, inducible expression, tissue- or cell-type-specific expression) on an operably linked nucleic acid sequence.

The selected promoter may be any DNA sequence that exhibits transcriptional activity in the selected host cell, and may be derived from a gene normally expressed in the host cell or from a gene normally expressed in other cells or organisms. Examples of promoters include, but are not limited to the following: A) prokaryotic promoters—E. coli lac, tac, or trp promoters, lambda phage P_{R} or P_{L} promoters, bacteriophage T7, T3, Sp6 promoters, B. subtilis alkaline protease promoter, and the B. stearothermophilus maltogenic amylase promoter, etc.; B) eukaryotic promoters—yeast promoters, such as GAL1, GAL4 and other glycolytic gene promoters (see for example, Hitzeman et al., 1980, J. Biol. Chem. 255: 12073-12080; Alber & Kawasaki, 1982, J. Mol. Appl. Gen. 1: 419-434), LEU2 promoter (Martinez-Garcia et al., 1989, Mol Gen Genet. 217: 464-470), alcohol dehydrogenase gene promoters (Young et al., 1982, in Genetic Engineering of Microorganisms for Chemicals, Hollaender et al., eds., Plenum Press, NY), or the TPI1 promoter (U.S. Pat. No. 4,599,311); insect promoters, such as the polyhedrin promoter (U.S. Pat. No. 4,745,051; Vasuvedan et al., 1992, FEBS Lett. 311: 7-11), the P10 promoter (Vlak et al., 1988, J. Gen. Virol. 69: 765-776), the Autographa californica polyhedrosis virus basic protein promoter (EP 397485), the baculovirus immediate-early gene promoter gene 1 promoter (U.S. Pat. Nos. 5,155,037 and 5,162,222), the baculovirus 39K delayed-early gene promoter (also U.S. Pat. Nos. 5,155,037 and 5,162,222) and the OpMNPV immediate early promoter 2; mammalian promoters—the SV40 promoter (Subramani et al., 1981, Mol. Cell. Biol. 1: 854-864), metallothionein promoter (MT-1; Palmiter et al., 1983, Science 222: 809-814), adenovirus 2 major late promoter (Yu et al., 1984, Nucl. Acids Res. 12: 9309-21), cytomegalovirus (CMV) or other viral promoter (Tong et al., 1998, Anticancer Res. 18: 719-725), or even the endogenous promoter of a gene of interest in a particular cell type.

A selected promoter may also be linked to sequences rendering it inducible or tissue-specific. For example, the addition of a tissue-specific enhancer element upstream of a selected promoter may render the promoter more active in a given tissue or cell type. Alternatively, or in addition, inducible expression may be achieved by linking the promoter to any of a number of sequence elements permitting induction by, for example, thermal changes (temperature sensitive), chemical treatment (for example, metal ion- or IPTG-inducible), or the addition of an antibiotic inducing agent (for example, tetracycline).

Regulatable expression is achieved using, for example, expression systems that are drug inducible (e.g., tetracycline, rapamycin or hormone-inducible). Drug-regulatable promoters that are particularly well suited for use in mammalian cells include the tetracycline regulatable promoters, and glucocorticoid steroid-, sex hormone steroid-, ecdysone-, lipopolysaccharide (LPS)- and isopropylthiogalactoside (IPTG)-regulatable promoters. A regulatable expression system for use in mammalian cells should ideally, but not necessarily, involve a transcriptional regulator that binds (or fails to bind) nonmammalian DNA motifs in response to a regulatory agent, and a regulatory sequence that is responsive only to this transcriptional regulator.

One inducible expression system that is well suited for the regulated expression of a GFP polypeptide of the invention or variant thereof, is the tetracycline-regulatable expression system, which is founded on the efficiency of the tetracycline resistance operon of E. coli. The binding constant between tetracycline and the tet repressor is high while the toxicity of tetracycline for mammalian cells is low, thereby allowing for regulation of the system by tetracycline concentrations in eukaryotic cell culture or within a mammal that do not affect cellular growth rates or morphology. Binding of the tet repressor to the operator occurs with high specificity.

Versions of the tet-regulatable system exist that allow either positive or negative regulation of gene expression by tetracycline. In the absence of tetracycline or a tetracycline analog, the wild-type bacterial tet repressor protein causes negative regulation of genes driven by promoters containing repressor binding elements from the tet operator sequences. Gossen & Bujard (1995, Science 268: 1766-1769; also International patent application No. WO 96/01313) describe a tet-regulatable expression system that exploits this positive regulation by tetracycline. In this system, tetracycline binds to a tet repressor fusion protein, rtTA, and prevents it from binding to the tet operator DNA sequence, thus allowing transcription and expression of the linked gene only in the presence of the drug.

This positive tetracycline-regulatable system provides one means of stringent temporal regulation of the GFP polypeptide of the invention or variant thereof (Gossen & Bujard, 1995, supra). The tet operator (tet 0) sequence is now well known to those skilled in the art. For a review, the reader is referred to Hillen & Wissmann (1989) in Protein-Nucleic Acid Interaction, “Topics in Molecular and Structural Biology”, eds. Saenger & Heinemann, (Macmillan, London), Vol. 10, pp 143-162. Typically the nucleic acid sequence encoding the GFP polypeptide is placed downstream of a plurality of tet O sequences: generally 5 to 10 such tet O sequences are used, in direct repeats.

In addition to the tetracycline-regulatable systems, a number of other options exist for the regulated or inducible expression of a GFP polypeptide or variant thereof according to the invention. For example, the E. coli lac promoter is responsive to lac repressor (lacI) DNA binding at the lac operator sequence. The elements of the operator system are functional in heterologous contexts, and the inhibition of lacI binding to the lac operator by IPTG is widely used to provide inducible expression in both prokaryotic, and more recently, eukaryotic cell systems. In addition, the rapamycin-controlled transcriptional activator system described by Rivera et al. (1996, Nature Med. 2: 1028-1032) provides transcriptional activation dependent on rapamycin. That system has low baseline expression and a high induction ratio.

Another option for regulated or inducible expression of a GFP polypeptide or variant thereof involves the use of a heat-responsive promoter. Activation is induced by incubation of cells, transfected with a GFP construct regulated by a temperature-sensitive transactivator, at the permissive temperature prior to administration. For example, transcription regulated by a co-transfected, temperature sensitive transcription factor active only at 37° C. may be used if cells are first grown at, for example, 32° C., and then switched to 37° C. to induce expression.

Tissue-specific promoters may also be used to advantage in GFP-encoding constructs of the invention. A wide variety of tissue-specific promoters is known. As used herein, the term “tissue-specific” means that a given promoter is transcriptionally active (i.e., directs the expression of linked sequences sufficient to permit detection of the polypeptide product of the promoter) in less than all cells or tissues of an organism. A tissue specific promoter is preferably active in only one cell type, but may, for example, be active in a particular class or lineage of cell types (e.g., hematopoietic cells). A tissue specific promoter useful according to the invention comprises those sequences necessary and sufficient for the expression of an operably linked nucleic acid sequence in a manner or pattern that is essentially the same as the manner or pattern of expression of the gene linked to that promoter in nature. The following is a non-exclusive list of tissue specific promoters and literature references containing the necessary sequences to achieve expression characteristic of those promoters in their respective tissues; the entire content of each of these literature references is incorporated herein by reference. Examples of tissue specific promoters useful with the R. Reniformis GFP of the invention are as follows: Bowman et al., 1995 Proc. Natl. Acad. Sci. USA 92, 12115-12119 describe a brain-specific transferrin promoter; the synapsin I promoter is neuron specific (Schoch et al., 1996 J. Biol. Chem. 271, 3317-3323); the necdin promoter is post-mitotic neuron specific (Uetsuki et al., 1996 J. Biol. Chem. 271, 918-924); the neurofilament light promoter is neuron specific (Charron et al., 1995 J. Biol. Chem. 270, 30604-30610); the acetylcholine receptor promoter is neuron specific (Wood et al., 1995 J. Biol. Chem. 270, 30933-30940); the potassium channel promoter is high-frequency firing neuron specific (Gan et al., 1996 J. Biol. Chem 271, 5859-5865); the chromogranin A promoter is neuroendocrine cell specific (Wu et al., 1995 A. J. Clin. Invest. 96, 568-578); the Von Willebrand factor promoter is brain endothelium specific (Aird et al., 1995 Proc. Natl. Acad. Sci. USA 92, 4567-4571); the flt-1 promoter is endothelium specific (Morishita et al., 1995 J. Biol. Chem. 270, 27948-27953); the preproendothelin-1 promoter is endothelium, epithelium and muscle specific (Harats et al., 1995 J. Clin. Invest. 95, 1335-1344); the GLUT4 promoter is skeletal muscle specific (Olson and Pessin, 1995 J. Biol. Chem. 270, 23491-23495); the Slow/fast troponins promoter is slow/fast twitch myofibre specific (Corin et al., 1995 Proc. Natl. Acad. Sci. USA 92, 6185-6189); the -Actin promoter is smooth muscle specific (Shimizu et al., 1995 J. Biol. Chem. 270, 7631-7643); the Myosin heavy chain promoter is smooth muscle specific (Kallmeier et al., 1995 J. Biol. Chem. 270, 30949-30957); the E-cadherin promoter is epithelium specific (Hennig et al., 1996 J. Biol. Chem. 271, 595-602); the cytokeratins promoter is keratinocyte specific (Alexander et al., 1995 B. Hum. Mol. Genet. 4, 993-999); the transglutaminase 3 promoter is keratinocyte specific (J. Lee et al., 1996 J. Biol. Chem. 271, 4561-4568); the bullous pemphigoid antigen promoter is basal keratinocyte specific (Tamai et al., 1995 J. Biol. Chem. 270, 7609-7614); the keratin 6 promoter is proliferating epidermis specific (Ramirez et al., 1995 Proc. Natl. Acad. Sci. USA 92, 4783-4787); the collagen 1 promoter is hepatic stellate cell and skin/tendon fibroblast specific (Houglum et al., 1995 J. Clin. Invest. 96, 2269-2276); the type X collagen promoter is hypertrophic chondrocyte specific (Long & Linsenmayer, 1995 Hum. Gene Ther. 6, 419-428); the Factor VII promoter is liver specific (Greenberg et al., 1995 Proc. Natl. Acad. Sci. USA 92, 12347-1235); the fatty acid synthase promoter is liver and adipose tissue specific (Soncini et al., 1995 J. Biol. Chem. 270, 30339-3034); the carbamoyl phosphate synthetase I promoter is portal vein hepatocyte and small intestine specific (Christoffels et al., 1995 J. Biol. Chem. 270, 24932-24940); the Na—K—Cl transporter promoter is kidney (loop of Henle) specific (Igarashi et al., 1996 J. Biol. Chem. 271, 9666-9674); the scavenger receptor A promoter is macrophages and foam cell specific (Horvai et al., 1995 Proc. Natl. Acad. Sci. USA 92, 5391-5395); the glycoprotein IIb promoter is megakaryocyte and platelet specific (Block & Poncz, 1995 Stem Cells 13, 135-145); the yc chain promoter is hematopoietic cell specific (Markiewicz et al., 1996 J. Biol. Chem. 271, 14849-14855); and the CD11b promoter is mature myeloid cell specific (Dziennis et al., 1995 Blood 85, 319-329).

Any tissue specific transcriptional regulatory sequence known in the art may be used to advantage with a vector encoding R. reniformis GFP or a variant thereof.

In addition to promoter/enhancer elements, vectors useful according to the invention may further comprise a suitable terminator. Such terminators include, for example, the human growth hormone terminator (Palmiter et al., 1983, supra), or, for yeast or fungal hosts, the TPI1 (Alber & Kawasaki, 1982, supra) or ADH3 terminator (McKnight et al., 1985, EMBO J. 4: 2093-2099).

Vectors useful according to the invention may also comprise polyadenylation sequences (e.g., the SV40 or Ad5E1b poly(A) sequence), and translational enhancer sequences (e.g., those from Adenovirus VA RNAs). Further, a vector useful according to the invention may encode a signal sequence directing the recombinant polypeptide to a particular cellular compartment or, alternatively, may encode a signal directing secretion of the recombinant polypeptide.

Coordinate expression of different genes from the same promoter in a recombinant vector maybe achieved by using an IRES element, such as the internal ribosomal entry site of Poliovirus.

type 1 from pSBC-1 (Dirks et al., 1993, Gene 128: 247-9). Internal ribosome binding site (IRES) elements are used to create multigenic or polycistronic messages. IRES elements are able to bypass the ribosome scanning mechanism of 5′ methylated Cap-dependent translation and begin translation at internal sites (Pelletier and Sonenberg, 1988, Nature 334: 320-325). IRES elements from two members of the picanovirus family (polio and encephalomyocarditis) have been described (Pelletier and Sonenberg, 1988, supra), as well an IRES from a mammalian message (Macejak and Sarnow, 1991 Nature 353: 90-94). Any of the foregoing may be used in an R. reniformis GFP vector in accordance with the present invention.

IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. In this manner, multiple genes, one of which will be an R. reniformis GFP gene, can be efficiently expressed using a single promoter/enhancer to transcribe a single message. Any heterologous open reading frame can be linked to IRES elements. In the present context, this means any selected protein that one desires to express and any second reporter gene (or selectable marker gene). In this way, the expression of multiple proteins could be achieved, for example, with concurrent monitoring through GFP production.

A vector useful according to the invention can also comprise a selectable marker allowing the identification of a cell that has received a functional copy of the GFP-encoding gene construct. In its simplest form, the GFP sequence itself, linked to a chosen promoter can be considered a selectable marker, in that illumination of cells or cell lysates with the proper wavelength of light and measurement of emitted fluorescence at the expected wavelength allows detection of cells that express the GFP construct. In other forms, the selectable marker can comprise an antibiotic resistance gene, such as the neomycin, bleomycin, zeocin or phleomycin resistance genes, or it can comprise a gene whose product complements a defect in a host cell, such as the gene encoding dihydrofolate reductase (DHFR), or, for example, in yeast, the Leu2 gene. Alternatively, the selectable marker can, in some cases, be a luciferase gene or a chromogenic substrate-converting enzyme gene such as the β-galactosidase gene.

GFP-encoding sequences according to the invention may be expressed either as free-standing polypeptides or frequently as fusions with other polypeptides. It is assumed that one of skill in the art can, given the polynucleotide sequences disclosed herein (e.g., SEQ ID NO: 1) readily construct a gene comprising a sequence encoding R. reniformis GFP or a fluorescent variant thereof and a sequence comprising one or more polypeptides or polypeptide domains of interest. It is understood that the fusion of GFP coding sequences and sequences encoding a polypeptide of interest maintains the reading frame of all polypeptide sequences involved. As used herein, the term “polypeptide of interest” or “domain of interest” refers to any polypeptide or polypeptide domain one wishes to fuse to a GFP molecule of the invention. The fusion of a GFP polypeptide of the invention with a polypeptide of interest, i.e. a transactivation domain, can be through linkage of the GFP sequence to either the N or C terminus of the fusion partner. Fusions comprising GFP polypeptides of the invention need not comprise only a singel polypeptide or domain in addition to the GFP. Rather, any number of domains of interest may be linked in any way as long as the GFP coding region retains its reading frame and the encoded polypeptide retains fluorescence activity under at least one set of conditions. One non-limiting example of such conditions includes physiological salt concentration (i.e., about 90 mM), pH near neutral and 37° C.

a. Plasmid Vectors.

Any plasmid vector that allows expression of a GFP coding sequence of the invention in a selected host cell type is acceptable for use according to the invention. A plasmid vector useful in the invention may have any or all of the above-noted characteristics of vectors useful according to the invention. Plasmid vectors useful according to the invention include, but are not limited to the following examples: Bacterial—pQE70, pQE60, pQE-9 (Qiagen) pBs, phagescript, psiX174, pBluescript SK, pBsKS, pNH8a, pNH16a, pNH18a, pNH46a (Stratagene); pTrc99A, pKK223-3, pKK233-3, pDR540, and pRIT5 (Pharmacia); Eukaryotic—pWLneo, pSV2cat, pOG44, pXT1, pSG (Stratagene) pSVK3, pBPV, pMSG, and pSVL (Pharmacia). However, any other plasmid or vector may be used as long as it is replicable and viable in the host.

b. Bacteriophage Vectors.

There are a number of well known bacteriophage-derived vectors useful according to the invention. Foremost among these are the lambda-based vectors, such as Lambda Zap II or Lambda-Zap Express vectors (Stratagene) that allow inducible expression of the polypeptide encoded by the insert. Others include filamentous bacteriophage such as the M13-based family of vectors.

c. Viral Vectors.

A number of different viral vectors are useful according to the invention, and any viral vector that permits the introduction and expression of sequences encoding R. reniformis GFP or variants thereof in cells is acceptable for use in the methods of the invention. Viral vectors that can be used to deliver foreign nucleic acid into cells include but are not limited to retroviral vectors, adenoviral vectors, adeno-associated viral vectors, herpesviral vectors, and Semliki forest viral (alphaviral) vectors. Defective retroviruses are well characterized for use in gene transfer (for a review see Miller, A. D. (1990) Blood 76: 271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14, and other standard laboratory manuals. Details of retrovirus production and host cell transduction of use in the methods of the invention are also presented in Example 1, below.

In addition to retroviral vectors, Adenovirus can be manipulated such that it encodes and expresses a gene product of interest but is inactivated in terms of its ability to replicate in a normal lytic viral life cycle (see for example Berkner et al., 1988, BioTechniques 6: 616; Rosenfeld et al., 1991, Science 252: 431-434; and Rosenfeld et al., 1992, Cell 68: 143-155). Suitable adenoviral vectors derived from the adenovirus strain Ad type 5 dl324 or other strains of adenovirus (e.g., Ad2, Ad3, Ad7 etc.) are well known to those skilled in the art. Adeno-associated virus (AAV) is a naturally occurring defective virus that requires another virus, such as an adenovirus or a herpes virus, as a helper virus for efficient replication and a productive life cycle. (For a review see Muzyczka et al., 1992, Curr. Topics in Micro. and Immunol. 158: 97-129). An AAV vector such as that described in Traschin et al. (1985, Mol. Cell. Biol. 5: 3251-3260) can be used to introduce nucleic acid into cells. A variety of nucleic acids have been introduced into different cell types using AAV vectors (see, for example, Hermonat et al., 1984, Proc. Natl. Acad. Sci. USA 81: 6466-6470; and Traschin et al., 1985, Mol. Cell. Biol. 4: 2072-2081).

Finally, the introduction and expression of foreign genes is often desired in insect cells because high level expression may be obtained, the culture conditions are simple relative to mammalian cell culture, and the post-translational modifications made by insect cells closely resemble those made by mammalian cells. For the introduction of foreign DNA to insect cells, such as Drosophila S2 cells, infection with baculovirus vectors is widely used. Other insect vector systems include, for example, the expression plasmid pIZ/V5-His (InVitrogen) and other variants of the pIZ/V5 vectors encoding other tags and selectable markers. Insect cells are readily transfectable using lipofection reagents, and there are lipid-based transfection products specifically optimized for the transfection of insect cells (for example, from PanVera).

2. Host Cells Useful According to the Invention.

Any cell into which a recombinant vector carrying an R. reniformis GFP or variant thereof can be introduced, and wherein the vector is permitted to drive the expression of the GFP or GFP variant sequence, is useful according to the invention. That is, because of the wide variety of uses for the GFP molecules of the invention, any cell in which a GFP molecule of the invention may be expressed and preferably detected is a suitable host. Vectors suitable for the introduction of GFP-encoding sequences to host cells from a variety of different organisms, both prokaryotic and eukaryotic, are described herein above or known to those skilled in the art.

Host cells can be prokaryotic, such as any of a number of bacterial strains, or can be eukaryotic, such as yeast or other fungal cells, insect or amphibian cells, or mammalian cells including, for example, rodent, simian or human cells. Cells expressing GFPs of the invention can be primary cultured cells, for example, primary human fibroblasts or keratinocytes, or can be an established cell line, such as NIH3T3, 293T or CHO cells. Further, mammalian cells useful for expression of GFPs of the invention can be phenotypically normal or oncogenically transformed. It is assumed that one skilled in the art can readily establish and maintain a chosen host cell type in culture.

3. Introduction of GFP-Encoding Vectors to Host Cells.

GFP-encoding vectors can be introduced to selected host cells by any of a number of suitable methods known to those skilled in the art. For example, GFP constructs may be introduced to appropriate bacterial cells by infection, in the case of E. coli bacteriophage vector particles such as lambda or M13, or by any of a number of transformation methods for plasmid vectors or for bacteriophage DNA. For example, standard calcium-chloride-mediated bacterial transformation is still commonly used to introduce naked DNA to bacteria (Sambrook et al., 1989, Molecular Cloning, A Laboratory Manual, Cold Spring Harbor Laboratory Press, Cold Spring Harbor, N.Y.), but electroporation may also be used (Ausubel et al., 1989, supra).

For the introduction of GFP-encoding constructs to yeast or other fungal cells, chemical transformation methods are generally used (e.g. as described by Rose et al., 1990, Methods in Yeast Genetics, Cold Spring Harbor Laboratory Press, Cold Spring Harbor, N.Y.). For transformation of S. cerevisiae, for example, the cells are treated with lithium acetate to achieve transformation efficiencies of approximately 10⁴ colony-forming units (transformed cells)/μg of DNA. Transformed cells are then isolated on selective media appropriate to the selectable marker used. Alternatively, or in addition, plates or filters lifted from plates may be scanned for GFP fluorescence to identify transformed clones.

For the introduction of R. reniformis GFP-encoding vectors to mammalian cells, the method used will depend upon the form of the vector. For plasmid vectors, DNA encoding R. reniformis GFP or variants thereof can be introduced by any of a number of transfection methods, including, for example, lipid-mediated transfection (“lipofection”), DEAE-dextran-mediated transfection, electroporation or calcium phosphate precipitation. These methods are detailed, for example, in Ausubel et al., 1989, supra.

Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™(Stratagene) kits are available. Other companies offering reagents and methods for lipofection include Bio-Rad Laboratories, CLONTECH, Glen Research, InVitrogen, JBL Scientific, MBI Fermentas, PanVera, Promega, Quantum Biotechnologies, Sigma-Aldrich, and Wako Chemicals USA.

For the introduction of R. reniformis GFP-encoding vectors to insect cells, such as Drosophila Schneider 2 cells (S2) cells, Sf9 or Sf21cells, transfection is also performed by lipofection.

Following transfection with an R. reniformis GFP-encoding vector of the invention, eukaryotic (preferably, but not necessarily mammalian) cells successfully incorporating the construct (intra- or extrachromosomally) can be selected, as noted above, by either treatment of the transfected population with a selection agent, such as an antibiotic whose resistance gene is encoded by the vector, or by direct screening using, for example, FACS of the cell population or fluorescence scanning of adherent cultures. Frequently, both types of screening are used, wherein a negative selection is used to enrich for cells taking up the construct and FACS or fluorescence scanning is used to further enrich for cells expressing GFPs or to identify specific clones of cells, respectively. For example, a negative selection with the neomycin analog G418 (Life Technologies, Inc.) can be used to identify cells that have received the vector, and fluorescence scanning can be used to identify those cells or clones of cells that express the R. reniformis GFP or GFP variant to the greatest extent.

II. How To Use R. reniformis GFP and Variants Thereof According to the invention.

R. reniformis GFP and variants thereof according to the invention are useful in a number of different ways. R. reniformis GFP has superior spectral characteristics and fluorescent intensity, relative to other GFPs, thus R. reniformis GFP is also useful in processes and assays beyond those that have previously been performed with other GFPs, such as Aequorea Victoria, Renilla mulleri and Ptilosarcus gurney.

A. The use of R. reniformis GFP for In Vivo Display of Peptide Libraries

R. reniformis GFP and variants thereof according to the invention are particularly useful for the in vivo display of peptide libraries in order to ascertain protein-protein or protein-nucleic acid interactions and to identify peptides that confer phenotypes of interest. Identification of peptides that exhibit particular phenotypes aid in the development of both therapeutic compounds and biological reagents that can be used for the “knock out” or modification of a given phenotype. There are many established screening assays known by those in the art that are designed to identify agents or compounds that inhibit particular disease states. Several examples of disease states that have suitable screening assays for therapeutic agent identification have already been described in U.S. patent application 2001/0003650 and are incorporated herein by reference. In essence, any established screening assay known in the art for a given phenotype is useful in the present invention. In addition, any assay that has been developed in the art that uses in vivo peptide libraries to identify protein-protein, and protein-nucleic acid interactions can be used herein.

1. Two-Hybrid Systems

A variety of biochemical procedures have been developed to identify interactions between proteins. One approach is the yeast two-hybrid system, an in vivo genetic approach to detect protein-protein interactions, originally described by Fields and Song (Nature 340: 245-246, 1989). The classical two-hybrid system can be applied to detect the interaction between two proteins (Fields and Song, 1989, supra) or to isolate interacting proteins from a library (“prey”) using a specific “bait” (Chien et al., (1991) Proc. Natl. Acad. Sci. USA 88: 9578-9582). In addition, the application of the two-hybrid system to an entire genome as either the bait or prey is being used to create protein linkage maps which catalog the network of interactions of an organism's complete proteome (Bartel et al., (1996) Nat. Genet. 12: 72-77).

There are several systems now used in the field of protein-protein interactions known by the following terms: two-hybrid, three-hybrid, tri-hybrid and tribrid, and reverse two hybrid. Each of these are systems for which the hrGFPs of the invention can be useful. There also exist modifications of each of these systems. Herein, the term “two-hybrid” is used to describe the classical bait and prey combination of Fields and Song as well as library screens described herein.

The yeast two-hybrid system is a genetic approach, which permits one to detect protein-protein interaction in vivo through the reconstitution of the activity of a transcriptional activator, such as GAL4, in yeast Saccharomyces cerevisiae. The key of the two-hybrid system is the finding that site-specific transcription factors are often modular, comprised of separable DNA-binding domains (BDs) that bind to a specific promoter sequence, and activation domains (ADs) that direct the RNA polymerase II complex to transcribe the gene downstream of the DNA binding site. This phenomenon is exploited by fusing separate binding and activation domains to a pair of interacting proteins, X and Y, to create two hybrid proteins, BD-X and AD-Y. Thus, generally any pair of DNA-binding domain and activation domain can be used. Furthermore, any site-specific transcription factor that has separable DNA binding domain and activation domain can be used. Co-expression of these two hybrids in a yeast cell leads to expression of a reporter gene containing the cognate BD-binding site. This approach can be also used to isolate cDNAs encoding partners for a protein of interest from an AD-Y library.

The two-hybrid system is advantageous over other biochemical methods for a number of reasons. The two-hybrid system permits an in vivo identification of the interacting proteins. Hence, the conformation of the target protein in yeast cells is probably closer to the native form than most of the in vitro conditions that are available, and it is therefore more likely to yield physiologically significant proteins. It is likely to be more sensitive for detection of protein-protein interaction than many other methods, such as probing an expression library with a labeled protein or co-immunoprecipitation, based on the parallel comparisons (Li et al. (1993) FASEB J. 7: 957-963). This sensitivity allows the isolation of weaker or transiently interacting proteins. Numerous protein interactions have been successfully detected by using the two-hybrid system, including cell cycle factors, signal transduction factors, and proteins involved in apoptosis and DNA repair.

The yeast two-hybrid system was developed to detect bimolecular interactions between two proteins in yeast. One of the limitations of this approach has been the inability to reconstitute interactions mediated by several components or interactions that are dependent on specific post-translational modifications which are not employed in yeast. Several assays have been described to overcome this barrier, including co-expression of a protein tyrosine kinase as a modifying enzyme to assay the interactions between phosphoproteins (Osborne et al. (1995) Bio/Technology 13: 1474-1478), introduction of adapter or ligand bridges to assay complex ternary interactions (Licitra & Liu, (1996) Proc. Natl. Acad. Sci. USA 93: 12817-12821; Zhang & Lauter (1996) Anal. Biochem. 242: 68-72) and assay of RNA-protein interactions (Putz et al. (1996) Nucleic Acids Res. 24: 4838-4840; Sen Gupta et al. (1996) Proc. Natl. Acad. Sci. USA 93: 8496-8501). All of these studies focus on a single bait protein and the interactions in the presence of the third protein, either as a modifier or stabilizer.

The yeast-two hybrid system has successfully been used to identify peptides that inhibit the yeast pheromone response (Caponigro et al. (1998) Proc. Natl. Acad. Sci. USA 95: 7508-7513). Further, methods for detecting multiple protein interactions have been described in U.S. Pat. Nos. 5,928,868 and 6,303,310.

A reverse two-hybrid system has also been established wherein molecules that disrupt protein complexes are identified (Vidal M et al. Proc Natl Acad Sci USA 1996 93: 10315-10320).

A mammalian two-hybrid system is equally useful in the present invention. Post-translational modifications etc. that are not normally present in yeast may be employed in mammalian cells (Dang, C. V., et al. (1991) Mol. Cell. Biol. 11: 954-962.) and thus result in biologically significant interactions.

In a preferred embodiment, a yeast two hybrid system that is based on the original interaction trap system (Gyrus et al. (1993) Cell 75: 791-803) will be used. In the interaction trap system, the protein of interest is expressed as a LexA fusion in a yeast strain containing LexA binding sites upstream of the selectable marker gene LEU2. A DNA library that encodes proteins fused to a transcription activation domain is introduced into the yeast strain. Cells that contain a library peptide that interacts with the known protein will grow on media lacking leucine since, the interaction allows for the transcriptional activation of LEU2. The yeast strain also contains a LexA operator-lacZ reporter and the amount of beta-galactosidase activity produced is a measure of the strength of the interaction. Colas et al. (Nature, 11: 548-550, 1996) has successfully used this system in the genetic selection of peptide aptamers, peptides that are scaffolded and anchored at both their amino and carboxy termini. A protein library was displayed by an E. coli thioredoxin-based scaffold that is fused to a modified set of protein moieties from the original interaction trap yeast two-hybrid system (LaVallie et al. (1993) Biotechnology 11: 187-193; Colas et al. (1996) Nature, 11: 548-550; Fabrizio et al. (1999) Oncogene, 18: 4357-4363).

To use the hybrid systems described herein, a transactivation domain is fused to the amino-, or carboxy-terminal end of hrGFP using standard molecular biology techniques and an expression cassette vector is generated wherein randomized peptides can be fused internally into hrGFP. In one embodiment, the transactivation protein that is used contains an SV40 nuclear localization signal, a B112 transcription activation domain, and a haemagglutinin epitope tag (Colas et al. Nature, 11: 548-550, 1996). The ability of the resulting hrGFP to transactivate can be tested using the interaction trap yeast two-hybrid system described above (Colas et al. Nature, 11: 548-550, 1996) and two known interacting protein partners.

The activation domain and DNA binding domain used in the hybrid assay can also be from a wide variety of transcriptional activator proteins that have separable binding and transcriptional activation domains. Examples include, but are not limited to, the GAL4 protein of S. cerevisiae, the GCN4 protein of S. cerevisiae (Hope and Struhl, (1986) Cell 46: 885-894), the ARD1 protein of S. cerevisiae (Thukral et al., (1989) Mol. Cell. Biol. 9: 2360-2369), and the human estrogen receptor (Kumar et al., (1987) Cell 51: 941-951). The DNA binding domain and activation domain which are incorporated into the fusion proteins do not need to be from the same transcriptional activator. It is preferred that the DNA binding domain and the transcription activator domain have nuclear localization signals (see Ylikomi et al., (1992) EMBO J. 11: 3681-3694; Dingwall and Laskey, (1991) TIBS 16: 479-481).

The reporter gene used in the assay contains the sequence encoding a detectable or selectable marker, the expression of which is regulated by the transcriptional activator. As used herein the term “regulated by” means that the expression of the reporter gene is increased by at least 10% and the expression varies with the activity of the transcriptional activator. The detectable or selectable marker is either turned on or off in the cell in response to the presence of a specific interaction. Preferably, the assay is carried out in the absence of background levels of the transcriptional activator (e.g., in a cell that is mutant or otherwise lacking in the transcriptional activator). In one embodiment, more than one reporter gene is used to detect transcriptional activation, e.g., LacZ and LEU2. The detectable marker can be any molecule that can give rise to a detectable signal, for example, detectable by antibody, enzymatic assay or fluorescence. A suitable selectable marker is any protein molecule that confers ability of a cell to grow under conditions that do not support the growth of cells in the absence of the selectable marker. For example, the selectable marker can be an enzyme that provides an essential nutrient. The reporter gene is operably linked to a promoter that contains a binding site for the DNA binding domain of the transcriptional activator. The reporter gene can either be under the control of the native promoter that naturally contains a binding site for the DNA binding protein, or under the control of a heterologous or synthetic promoter.

The host cell in which the interaction assay occurs can be any cell, prokaryotic or eukaryotic including, but not limited to, mammalian, bacteria, insect cells, and yeast cells. The cell must support transcription of the reporter gene and allow for its detection, The host cell used should not express an endogenous transcription factor that binds to the same DNA site as that recognized by the DNA binding domain fusion population. The host cell can also be a mutant that lacks an endogenous, functional form of the reporter gene(s) used in the assay. Suitable yeast host strains are known in the art and can be used in the method described herein (see, e.g., Bartel et al., (1993) “Using the two-hybrid system to detect protein-protein interactions,” in Cellular Interactions in Development, Hartley, D. A. (ed.), Practical Approach Series xviii, IRL Press at Oxford University Press, New York, N.Y., pp. 153-179; Fields and Sternglanz, (1994) TIG 10: 286-292).

The use of the R. reniformis as a GFP scaffold for the in vivo display of peptides in the hybrid systems has the particular advantage that the diversity of the library can be easily estimated by monitoring GFP autofluorescence and the expression of a displayed peptide can be monitored on a per cell basis.

2. Transdominant Protein-Protein Interactions

Peptide display libraries according to the invention are also useful in transdominant genetic experiments for identifying inhibitory, “knock out” protein molecules. Any assay known in the art that is used to identify dominant negative proteins can be used in the present invention (assays are described by, for example, Dang, C. V., et al. (1991) Mol. Cell. Biol. 11: 954-962; Holzmayer, T. A., et al., (1992) Nucl. Acids. Res., 20: 711-717; Whiteway, M., et al., (1992) Proc. Natl. Acad. Sci. USA, 89: 9410-9414; Gudkov, A. V., et al., (1994), Proc. Natl. Acad. Sci. USA, 91: 3744-3748; Herskowitz, I., (1987), Nature (London)(London), 329: 219-222; Ramer, S. W., et al., (1992), Proc. Natl. Acad. Sci. USA, 89: 11589-11593; Edwards, M. C., et al., (1997), Genetics, 147: 1063-1076 and U.S. Pat. Nos. 5,955,275 and 6,025,485). Basically, the hrGFP scaffold peptide library is introduced into host cells and a specific selection criteria for an altered phenotype is enforced. Cells exhibiting the selected altered phenotype are then used to isolate the coding sequence for peptides of interest, for example by PCR. The peptides and their targets can be then further characterized to determine at what stage within a particular biochemical pathway the peptides act. For example, a particular target molecule may be confirmed by yeast-two hybrid analysis.

A reporter gene construct can be used as a reporter for a particular phenotype. The reporter construct is chosen carefully to represent the relevant phenotype as closely as possible. The reporter gene, for example, can be placed under the control of a promoter that is only active during the relevant state. A reporter gene is expressed at such levels that it can be detected quantitatively and it enables the rapid selection of cells that exhibit an altered phenotype. Suitable reporter genes for the present invention include, but are not limited to the LacZ gene, the CAT gene and the luciferase gene, and can also include genes for proteins that are expressed on the cell surface.

The phenotypes of interest can also be detected by any other means known in the art and the assay will be dependent upon the phenotype to be measured. For example, change in membrane potentials can be monitored by patch-clamp techniques, morphological changes by microscopic analysis, changes in molecule expression by western, northern, Southern, PCR, immunohistochemistry, or FACS analysis etc. Susceptibility of cells to pathogens can be monitored by cell viability assays, syncytial assays, or any other standard assay used to monitor pathogenic infection. In addition, reporter cells may be used. For example, a second cell may respond to a signal provided by a first cell exhibiting the phenotype of interest. The use of peptide libraries to identify peptides that disrupt biochemical pathways has been described in WO 98/39483A1, which is incorporated herein by reference. Further, there are several examples of assays known in the art that are used for the identification of cytokine, hormone and growth factor signaling pathway agonists and antagonists. (For example, those found in U.S. Pat. Nos. 6,312,941, 6,232,081, 6,210,913, also incorporated herein by reference).

Once a displayed peptide is found to alter a given phenotypic response, the sequence of the peptide can be used to generate additional candidate peptides with the same function, for example by using the mutagenesis assays described herein. The identified peptide can also be used to pull out target molecules by using the peptide as “bait” in yeast or mammalian two hybrid systems or by co-immunoprecipitation, etc. Alternatively, molecular biological techniques can be used to screen expression libraries by using the identified peptide as a probe.

3. Identification of Peptides for Treatment of Pathogenic Diseases

A wide variety of screening methods for compounds or agents that inhibit pathogenic diseases have been established and are known to those skilled in the art. Often the screening method identifies agents that block constitutively active signal transduction pathways, apoptosis, specific protein-protein interactions, cytokine production, pathogenic infection, or a particular protein modification.

For example, the hrGFP-scaffolded peptide library can be used to screen for peptides that inhibit the growth of tumor cells. The library can be introduced into either primary or immortalized tumor cells to identify peptides that inhibit cell growth and/or induce apoptosis. Alternatively, non-cancerous, healthy cells can be transformed using known oncogenes. Upon introduction of a library according to the invention, peptides can be identified that reverse the transformed state. The are many assays known in the art for the detection of transformed states and their inhibition (e.g. soft agar and membrane ruffling assays).

hrGFP-scaffolded peptides can be further screened for their ability to block signal transduction pathways involved in tumorgenisis and metastasis. For example, hrGFP-scaffolded peptides can be screened for peptides that block platelet derived growth factor or epidermal growth factor signaling. In the case of metastisis, peptides that block molecules involved in invasion, for example, RAS, v-mos, v-raf, v-src, and v-FES are of particular interest.

The hrGFP-scaffolded peptide libraries described herein can also be used to screen for peptides that inhibit replication of, or initial infection by, an infectious agent. Several assays are well known in the art. For example, assays have been developed to identify compounds or agents that inhibit HIV entry, including syncytia formation and reporter gene assays. In addition, screening methods to identify agents that inhibit hepatitis C virus replication have been established (U.S. Pat. No. 6,326,151), as well as screening methods for identifying anti-fungal agents (U.S. Pat. Nos. 6,277,564 and 6,117,641).

### EXAMPLES

The invention will now be further illustrated with reference to the following examples. It will be appreciated that what follows is by way of example only and that modifications to detail may be made while still falling within the scope of the invention.

### Example 1

### Construction of a R. reniformis hrGFP Insertion Mutant

Isolation of peptide inhibitors of intracellular processes is important for drug design, research target identification, and validation of microarray hits. Unlike chemical reagents, peptides offer the potential for in vivo expression and target screening within the intracellular environment. However, peptides are sensitive to proteolytic degradation and exist in numerous conformations in aqueous solution. Expression of peptides as a fusion to stable proteins reduces the probability that the peptide will be degraded, stabilizes peptide conformation, and increases the peptide's affinity for potential binding targets. While a number of protein scaffolds have been described, green fluorescent protein offers the advantage of being easily monitored by fluorescence microscopy or fluorescent activated cell sorting (FACS).

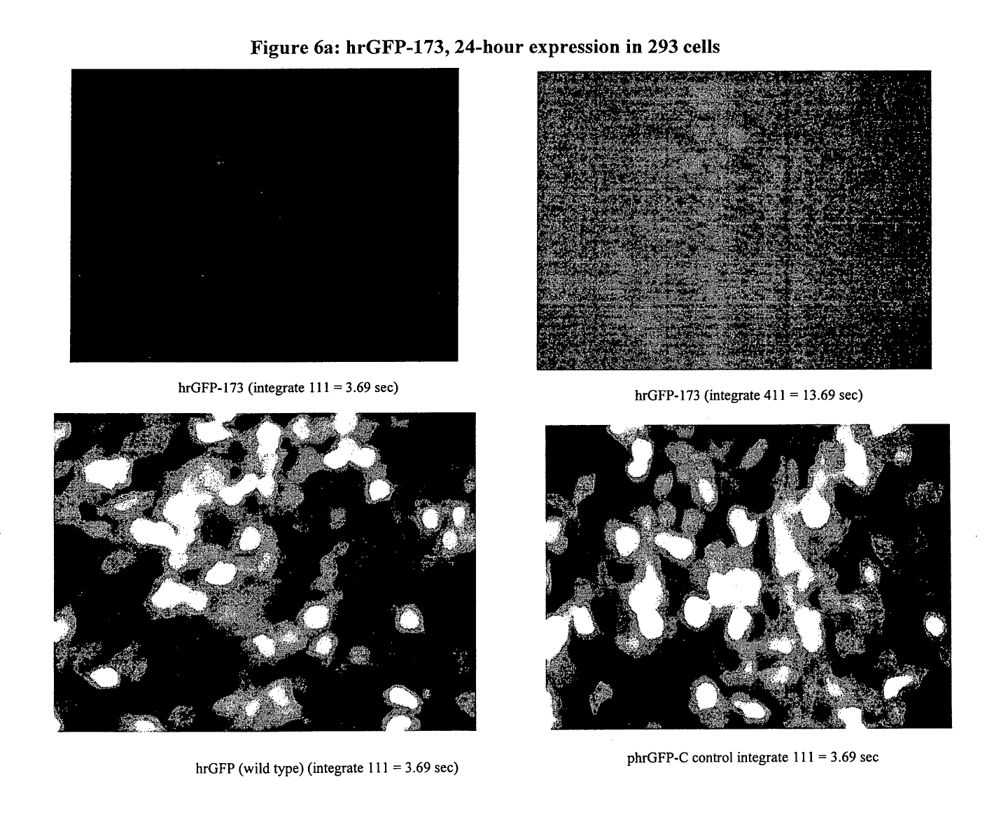

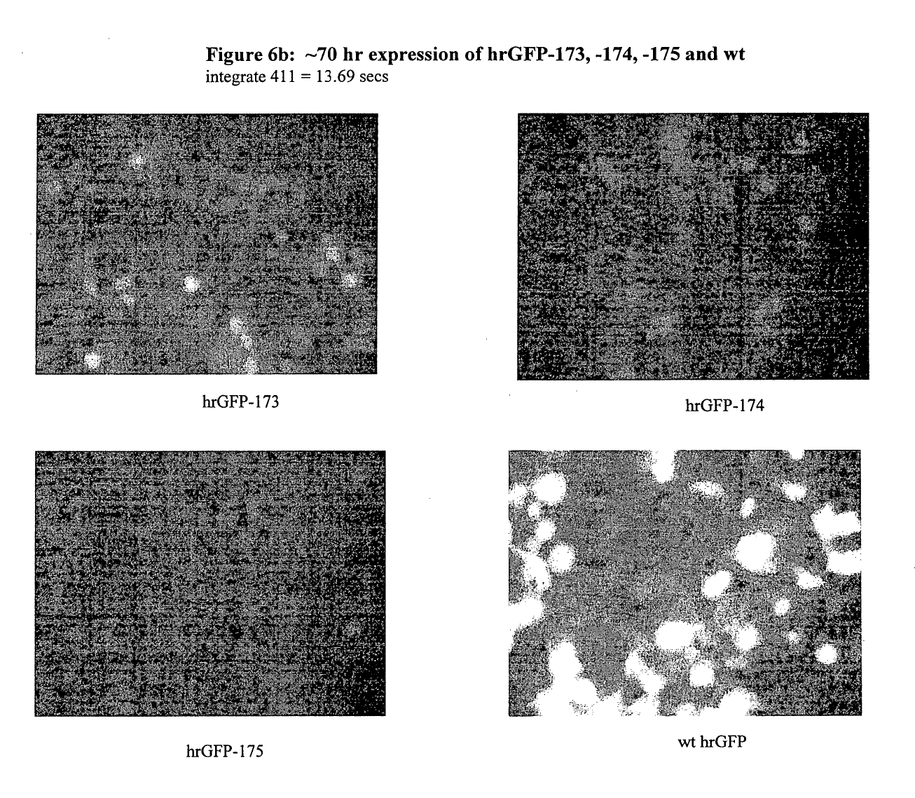

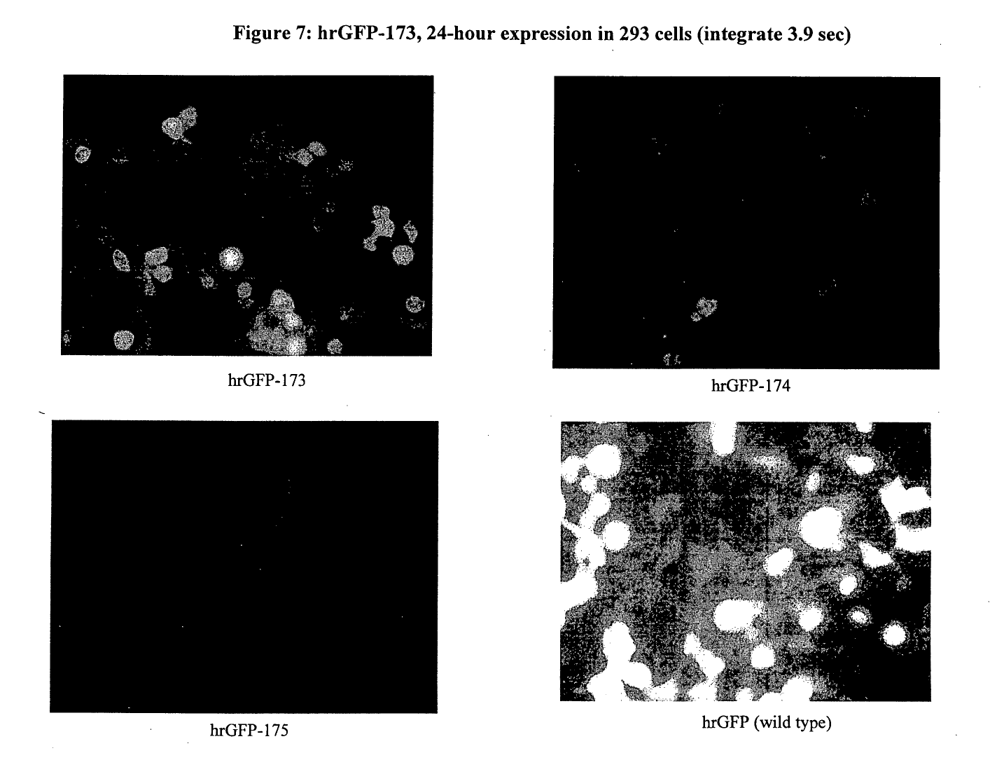

Green fluorescent protein from humanized Renilla Reniformis (hrGFP) is tolerant to insertion of peptides. In particular, an 18 base pair multiple cloning site sequence has been inserted between nucleotides 519 and 520 of hrGFP. The sequence encodes a hrGFP protein with a six amino acid insert between amino acids 173 and 174 of wild type hrGFP. As assessed by fluorescence microscopy, hrGFP-173 fluoresces in 293 cells within 24 h after transfection of the hrGFP-173 gene (FIG. 6A). The hrGFP-173 insertion mutant qualitatively produces more fluorescence in comparison to wild-type hrGFP than hrGFP-174 and hrGFP 175 (FIG. 7).

Construction of hrGFP-173

Construction of the hrGFP-173 gene was performed by PCR using two sets of primers in two separate PCR reactions:

Set 1:
N-GFP5′Kozak:

(SEQ ID NO: 9)

5′-ATTATTGCGGCCGCATCCACCATGGTGAGCAAGCAGATC-3′
GFP-5′-173:

(SEQ ID NO: 10)

5′-ATTATTGAATTCGACGTCGGCAAGTTCTACAGCTGCCAC-3′

Set 2:
GFP3′-173:

(SEQ ID NO: 11)

5′-ATTATTGAATTCAGATCTGCTGTTCAGGCGGTACACCA-3′
X-GFP3′:

(SEQ ID NO: 12)

5′-ATTATTATTCTCGAGCTATTACACCCACTCGTGCAGG-3′

The product of the Set 1 PCR reaction was a fragment of 558 base pairs consisting of the first 519 base pairs of hrGFP flanked at the 5′ end by a NotI restriction site and at the 3′ end by BglII and EcoRI sites. The product of the Set 2 PCR reaction was a fragment of 237 base pairs consisting of the last 201 base pairs hrGFP (including the stop codon) flanked at the 5′ end by EcoRI and AatII sites and at the 3′ end by an XhoI site.

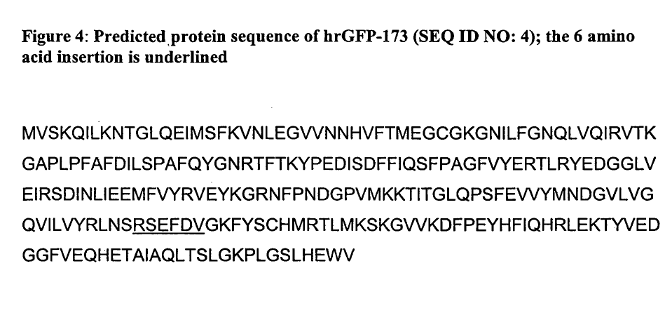

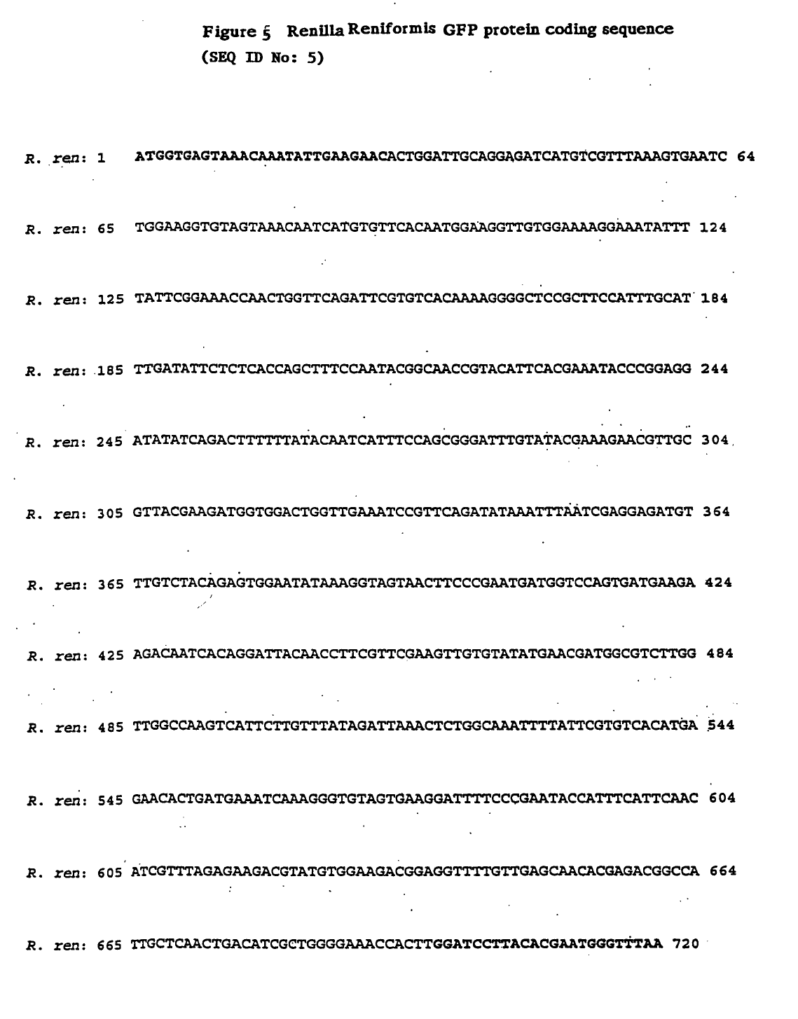

The two fragments were digested with EcoRI and ligated. The ligated product was amplified in a PCR reaction with N-GFP5′ and X-GFP3′ primers using Pfu polymerase (Stratagene). The resulting product was approximately 765 base pairs and consisted of the hrGFP gene with an 18 base pair insertion between bases 519 and 520, and flanked by NotI (5′) and XhoI (3′) sites. The 18 base pair insertion consisted of 5′-BglII-EcoRI-AatII-3′. The product was digested with NotI and XhoI and ligated into phrGFP-1 (Stratagene), cut with the same two enzymes. The resulting plasmid is referred to as phrGFP-173. phrGFP-173 was sequenced and shown to contain the expected insertion. The nucleic acid and polypeptide sequences of the hrGFP-173 sequence are shown in FIGS. 2 and 4, respectively.

Fluorescence Microscopy:

Upon expression, hrGFP-173 is predicted to produce a protein containing a 6 amino acid insert (R-S-E-F-D-V) between S173 and G174 (see FIG. 4). To determine if this six amino acid insert allows the protein to fold and fluoresce within cells, phrGFP-173 was transformed into 293 cells, and the fluorescence was examined under a fluorescence microscope (with a B2A filter) at 24 and 72 hours. Faint fluorescence was observed after 24 hours (FIG. 6a), and significant fluorescence was observed after 70 hours (FIG. 6b). In comparison, mutants containing the 18 base pair insert between amino acids 174/175, or 175/176 showed significantly reduced, or no fluorescence, after 70 hours (FIGS. 6b, 7). Wild type hrGFP expressed from plasmid phrGFP-C (Stratagene) and wild type hrGFP constructed by PCR with N-GFP5′ Kozak/X-GFP3′ primers showed bright fluorescence after 24 hours. A total of nine different insertion sites were tested along with hrGFP-173, including insertion following amino acids 41, 157, 172-175, 177, 178 and 192 (constructed and analyzed by methods similar to those outlined above). hrGFP-173 gave rise to the brightest fluorescence of all the mutants tested. Note that these data are qualitative in nature. Quantitative data will require computerized integration of microscopy data or FACS analysis.

The results demonstrate that hrGFP is tolerant to insertions between amino acids Ser-173 and Gly-174. While fluorescence of the hrGFP-173 mutant is reduced compared to wild type hrGFP, fluorescence is easily observed between 24-70 hours post-transfection. Therefore, this site can be used for insertion of random peptide libraries while minimizing hrGFP insolubility and loss of fluorescence. For use as a scaffold, hrGFP-173 must present peptides in a soluble form, stabilize the inserted peptide's conformation, and tolerate a wide variety of unique peptide sequences. Fluorescence activity of hrGFP-173 should permit the monitoring of peptide-scaffold expression and solubility, as well as facilitating screening of peptide library members.

The foregoing examples demonstrate experiments performed and contemplated by the present inventors in making and carrying out the invention. It is believed that these examples include a disclosure of techniques which serve to both apprise the art of the practice of the invention and to demonstrate its usefulness. It will be appreciated by those of skill in the art that the techniques and embodiments disclosed herein are preferred embodiments only that in general numerous equivalent methods and techniques may be employed to achieve the same result.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1. A recombinant polynucleotide comprising a first nucleic acid sequence encoding a humanized Renilla reniformis green fluorescent protein (hrGFP) and a second heterologous nucleic acid sequence inserted internally into said first nucleic acid sequence encoding humanized hrGFP, said recombinant polynucleotide encoding a scaffold GFP.

2. The recombinant polynucleotide of claim 1 wherein said scaffold GFP is fluorescent.

3. The recombinant polynucleotide of claim 1 wherein the said first nucleic acid sequence encoding a hrGFP is SEQ ID NO: 1.

4. The recombinant polynucleotide of claim 2 wherein said second heterologous nucleic acid sequence is inserted between nucleotides 519 and 520 of said first nucleic acid sequence encoding hrGFP.

5. The recombinant polynucleotide of claim 1 wherein said second heterologous nucleic acid sequence comprises a multiple cloning site sequence.

6. The recombinant polynucleotide of claim 1 wherein said second heterologous nucleic acid sequence is the multiple cloning site sequence of SEQ ID NO: 2.

7. The recombinant polynucleotide of claim 4 or 5 further comprising a third nucleic acid sequence inserted internally into said multiple cloning site, wherein said third nucleic acid sequence comprises a random nucleic acid sequence.

8. The recombinant polynucleotide of claim 6 wherein said third nucleic acid sequence encodes a peptide in frame with said hrGFP coding sequences.

9. The recombinant polynucleotide of claim 6 wherein said third nucleic acid sequence encodes a peptide of 2 to 50 amino acids.

10. The recombinant polynucleotide of claim 6 wherein said third nucleic acid sequence encodes a polypeptide of 10 to 20 amino acids.

11. A recombinant polypeptide comprising Renilla reniformis green fluorescent protein (GFP) and a heterologous peptide that is fused internally into said GFP.

12. The recombinant polypeptide of claim 7 wherein said heterologous peptide is located between amino acid residues 173 and 174 of said GFP.

13. The recombinant polypeptide of claim 7 wherein said second heterologous amino acid sequence is a random peptide sequence.

14. A recombinant vector comprising the recombinant polynucleotide sequence of any of claims 1-6.

15. The recombinant vector of claim 11 wherein said vector is selected from the group consisting of a plasmid, a bacteriophage, a virus, and a retrovirus.

16. A cell comprising the recombinant vector of claim 11.

17. A library of recombinant vectors comprising a plurality of recombinant polynucleotides wherein said recombinant polynucleotides comprise a first nucleic acid sequence encoding humanized Renilla reniformis green fluorescent protein (hrGFP) and a second heterologous nucleic acid sequence inserted internally into said first nucleic acid sequence encoding hrGFP, wherein the members of the library comprise a plurality of different said second heterologous nucleic acid sequences.

18. The library of claim 17 wherein said plurality of different said second heterologous nucleic acid sequences comprise a pluriality of randomized nucleic acid sequences.

19. A method for identifying a peptide conferring a phenotype of interest comprising the steps of:
a) providing a plurality of cells, each cell containing a recombinant vector comprising a recombinant polynucleotide that encodes a recombinant polypeptide comprising Renilla reniformis green fluorescent protein (hrGFP) and a heterologous random peptide wherein said heterologous random peptide is fused internally into said hrGFP, under conditions wherein said recombinant polypeptide is expressed; and
b) assaying said cells for said phenotype.

20. A method for identifying a peptide that interacts with a protein of interest, the method comprising the steps of:
a) introducing a library of recombinant vectors comprising recombinant polynucleotides that encode recombinant polypeptides into a plurality of host cells and maintaining said cells under conditions wherein said recombinant polypeptides are expressed,
wherein said recombinant polypeptides comprise Renilla reniformis green fluorescent protein (hrGFP) fused to a transactivation domain and a heterologous randomized peptide fused internally into said hrGFP and,
wherein said host cells contain a gene that encodes a protein of interest fused to a DNA binding domain, and a reporter gene functionally linked to a DNA sequence that binds said DNA binding domain, wherein expression of said reporter gene is regulated by said transactivation domain and;
b) detecting expression of said reporter gene, wherein detection of reporter gene expression identifies said heterologous random peptide as a peptide that interacts with the protein of interest.
